SEQUENCE LISTING (17 sequences)

SEQ ID NO: 1 - DNA, 20 bp, Artificial Sequence
  sgRNA sequence targeting wild type mF8 exon 1
gccatcagaagatactacct

SEQ ID NO: 2 - DNA, 20 bp, Artificial Sequence
  sgRNA targeting mutant mF8 exon 1
atcagaagatactaccttgg

SEQ ID NO: 3 - DNA, 56 bp, Mus musculus
atcagaagatactaccttggtgcagtggaattgtcctggaactatattcagagtga

SEQ ID NO: 4 - DNA, 51 bp, Artificial Sequence
  NSG HemA mouse mutant mF8 exon 1
atcagaagatactaccttggtggaattgtcctggaactatattcagagtga

SEQ ID NO: 5 - DNA, 62 bp, Mus musculus
agtgccatcagaagatactaccttggtgcagtggaattgtcctggaactatattcagagtga

SEQ ID NO: 6 - DNA, 57 bp, Artificial Sequence
  NSG HemA mouse mutant mF8 exon 1
agtgccatcagaagatactaccttggtggaattgtcctggaactatattcagagtga

SEQ ID NO: 7 - protein, 46 aa, Mus musculus
MQIALFACFFLSFLFNFCSSAIRRYYLGAVELSWNYIQSDLLSVLH

SEQ ID NO: 8 - protein, 37 aa, Artificial Sequence
  Mutant Factor VIII amino acids
MQIALFACFFLSFLFNFCSSAIRRYYLGGIVLELYSE

SEQ ID NO: 9 - protein, 45 aa, Artificial Sequence
  Indel repair Factor VIII amino acids; +2 repair
  misc_feature (29)..(29): Xaa can be any naturally occurring amino acid
MQIALFACFFLSFLFNFCSSAIRRYYLGXELSWNYIQSDLLSVLH

SEQ ID NO: 10 - protein, 44 aa, Artificial Sequence
  Indel repair Factor VIII amino acids; -1 repair
MQIALFACFFLSFLFNFCSSAIRRYYLGELSWNYIQSDLLSVLH

SEQ ID NO: 11 - DNA, 11703 bp, Artificial Sequence
  Cas9 nuclease targeting mF8 guide RNA
ttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccagagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccatcagaagatactacctgttttagagctagaaatagcaagttaaaataaggctagtccgttatcaacttgaaaaagtggcaccgagtcggtgcttttttgaattctagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggcgccggctcgagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcgggatccgccaccatggattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgtcctgctgctactaagaaagctggtcaagctaagaaaaagaaagctagcggcagcggcgccaccaacttcagcctgctgaagcaggccggcgacgtggaggagaaccccggccccatggtgtctaagggcgaagagctgattaaggagaacatgcacatgaagctgtatatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaagcgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagagacctacgtcgagcagcacgaggtggctgtggccagatactgcgacctccctagcaaactggggcacaaacttaattgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagc

SEQ ID NO: 12 - DNA, 11704 bp, Artificial Sequence
  Cas9 nuclease targeting NSGHA mF8 guide RNA
ttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccagagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgatcagaagatactaccttgggttttagagctagaaatagcaagttaaaataaggctagtccgttatcaacttgaaaaagtggcaccgagtcggtgcttttttgaattctagatcttgagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccactttggcgccggctcgagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgacgcgggatccgccaccatggattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgtcctgctgctactaagaaagctggtcaagctaagaaaaagaaagctagcggcagcggcgccaccaacttcagcctgctgaagcaggccggcgacgtggaggagaaccccggccccatggtgtctaagggcgaagagctgattaaggagaacatgcacatgaagctgtatatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgacttctttaagcagtccttccctgagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaagcgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagagacctacgtcgagcagcacgaggtggctgtggccagatactgcgacctccctagcaaactggggcacaaacttaattgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtacgtatagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagc

SEQ ID NO: 13 - protein, 2351 aa, Homo sapiens [sequence omitted]

SEQ ID NO: 14 - protein, 2319 aa, Mus musculus
MQIALFACFFLSLFNFCSSAIRRYYLGAVELSWNYIQSDLLSVLHTDSRFLPRMSTSFPFNTSIMYKKTVFVEYKDQLFNIAKPRPPWMGLLGPTIWTEVHDTVVITLKNMASHPVSLHAVGVSYWKASEGDEYEDQTSQMEKEDDKVFPGESHTYVWQVLKENGPMASDPPCLTYSYMSHVDLVKDLNSGLIGALLVCKEGSLSKERTQMLYQFVLLFAVFDEGKSWHSETNDSYTQSMDSASARDWPKMHTVNGYVNRSLPGLIGCHRKSVYWHVIGMGTTPEIHSIFLEGHTFFVRNHRQASLEISPITFLTAQTLLIDLGQFLLFCHISSHKHDGMEAYVKVDSCPEESQWQKKNNNEEMEDYDDDLYSEMDMFTLDYDSSPFIQIRSVAKKYPKTWIHYISAEEEDWDYAPSVPTSDNGSYKSQYLSNGPHRIGRKYKKVRFIAYTDETFKTRETIQHESGLLGPLLYGEVGDTLLIIFKNQASRPYNIYPHGITDVSPLHARRLPRGIKHVKDLPIHPGEIFKYKWTVTVEDGPTKSDPRCLTRYYSSFINPERDLASGLIGPLLICYKESVDQRGNQMMSDKRNVILFSIFDENQSWYITENMQRFLPNAAKTQPQDPGFQASNIMHSINGYVFDSLELTVCLHEVAYWHILSVGAQTDFLSIFFSGYTFKHKMVYEDTLTLFPFSGETVFMSMENPGLWVLGCHNSDFRKRGMTALLKVSSCDKSTSDYYEEIYEDIPTQLVNENNVIDPRSFFQNTNHPNTRKKKFKDSTIPKNDMEKIEPQFEEIAEMLKVQSVSVSDMLMLLGQSHPTPHGLFLSDGQEAIYEAIHDDHSPNAIDSNEGPSKVTQLRPESHHSEKIVFTPQPGLQLRSNKSLETTIEVKWKKLGLQVSSLPSNLMTTTILSDNLKATFEKTDSSGFPDMPVHSSSKLSTTAFGKKAYSLVGSHVPLNVSEENSDSNILDSTLMYSQESLPRDNILSMENDRLLREKRFHGIALLTKDNTLFKDNVSLMKTNKTYNHSTTNEKLHTESPTSIENSTTDLQDAILKVNSEIQEVTALIHDGTLLGKNSTYLRLNHMLNRTTSTKNKDIFHRKDEDPIPQDEENTIMPFSKMLFLSESSNWFKKTNGNNSLNSEQEHSPKQLVYLMFKKYVKNQSFLSEKNKVTVEQDGFTKNIGLKDMAFPHNMSIFLTTLSNVHENGRHNQEKNIQEEIEKEALIEEKVVLPQVHEATGSKNFLKDILILGTRQNISLYEVHVPVLQNITSINNSTNTVQIHMEHFFKRRKDKETNSEGLVNKTREMVKNYPSQKNITTQRSKRALGQFRLSTQWLKTINCSTQCIIKQIDHSKEMKKFITKSSLSDSSVIKSTTQTNSSDSHIVKTSAFPPIDLKRSPFQNKFSHVQASSYIYDFKTKSSRIQESNNFLKETKINNPSLAILPWNMFIDQGKFTSPGKSNTNSVTYKKRENIIFLKPTLPEESGKIELLPQVSIQEEEILPTETSHGSPGHLNLMKEVFLQKIQGPTKWNKAKRHGESIKGKTESSKNTRSKLLNHHAWDYHYAAQIPKDMWKSKEKSPEIISIKQEDTILSLRPHGNSHSIGANEKQNWPQRETTWVKQGQTQRTCSQIPPVLKRHQRELSAFQSEQEATDYDDAITIETIEDFDIYSEDIKQGPRSFQQKTRHYFIAAVERLWDYGMSTSHVLRNRYQSDNVPQFKKVVFQEFTDGSFSQPLYRGELNEHLGLLGPYIRAEVEDNIMVTFKNQASRPYSFYSSLISYKEDQRGEEPRRNFVKPNETKIYFWKVQHHMAPTEDEFDCKAWAYFSDVDLERDMHSGLIGPLLICHANTLNPAHGRQVSVQEFALLFTIFDETKSWYFTENVKRNCKTPCNFQMEDPTLKENYRFHAINGYVMDTLPGLVMAQDQRIRWYLLSMGNNENIQSIHFSGHVFTVRKKEEYKMAVYNLYPGVFETLEMIPSRAGIWRVECLIGEHLQAGMSTLFLVYSKQCQIPLGMASGSIRDFQITASGHYGQWAPNLARLHYSGSINAWSTKEPFSWIKVDLLAPMIVHGIKTQGARQKFSSLYISQFIIMYSLDGKKWLSYQGNSTGTLMVFFGNVDSSGIKHNSFNPPIIARYIRLHPTHSSIRSTLRMELMGCDLNSCSIPLGMESKVISDTQITASSYFTNMFATWSPSQARLHLQGRTNAWRPQVNDPKQWLQVDLQKTMKVTGIITQGVKSLFTSMFVKEFLISSSQDGHHWTQILYNGKVKVFQGNQDSSTPMMNSLDPPLLTRYLRIHPQIWEHQIALRLEILGCEAQQQY

SEQ ID NO: 15 - DNA, 38 bp, Artificial Sequence
  mF8 DNA in NSA HemA mice
gccatcagaagatactaccttggtggaattgtcctgga

SEQ ID NO: 16 - DNA, 38 bp, Artificial Sequence
  mF8 DNA in NSA HemA mice with indel mutation +5
gccatcagaagatactaccttggggtggtggaattgtc

SEQ ID NO: 17 - DNA, 37 bp, Artificial Sequence
  mF8 DNA in NSA HemA mice with indel mutation -1
gccatcagaagatactacctggtggaattgtcctgga

(19) United States
(12) Patent Application Publication
(10) Pub. No.: US 2024/0279649 A1
(43) Pub. Date: Aug. 22, 2024

(54) GENE EDITING FOR EXPRESSION OF FUNCTIONAL FACTOR VIII FOR THE TREATMENT OF HEMOPHILIA

(71) Applicant: Seattle Children's Hospital d/b/a Seattle Children's Research Institute, Seattle, WA (US)

(72) Inventor: Carol Hsing Miao, Seattle, WA (US)

(73) Assignee: Seattle Children's Hospital d/b/a Seattle Children's Research Institute, Seattle, WA (US)

(21) Appl. No.: 18/290,433

(22) PCT Filed: May 11, 2022
(86) PCT No.: PCT/US22/28843

§ 371 Date: Nov. 13, 2023

Related U.S. Application Data
(60) Provisional application No. 63/331,591, filed on Apr. 15, 2022.
(60) Provisional application No. 63/187,200, filed on May 11, 2021.

(51) Int. Cl. C12N 15/11; A61K 48/00; A61P 7/04; C12N 9/22; C12N 15/88

(52) U.S. Cl. CPC C12N 15/11; A61K 48/0041; A61K 48/005; A61P 7/04; C12N 9/22; C12N 15/88; C12N 2310/20

(57) ABSTRACT

Gene-editing to allow for expression of functional factor VIII for the treatment of hemophilia A is described. The disclosure further provides nanoparticles to deliver gene-editing components to liver sinusoidal endothelial cells (LSEC) to correct mutant factor VIII genes.

### CROSS-REFERENCE TO RELATED APPLICATION

This application is a U.S. National Phase Application based on International Patent Application No. PCT/US2022/028843, filed on May 11, 2022, which claims priority to U.S. Provisional Patent Application No. 63/187,200 filed on May 11, 2021 and U.S. Provisional Patent Application No. 63/331,591 filed on April 15, 2022, each of which are incorporated herein by reference in their entirety as if fully set forth herein.

### STATEMENT REGARDING FEDERALLY SPONSORED RESEARCH OR DEVELOPMENT

This invention was made with government support under grant HL151077 awarded by the National Institutes of Health. The government has certain rights in the invention.

### REFERENCE TO SEQUENCE LISTING

The Sequence Listing associated with this application is provided in text format in lieu of a paper copy and is hereby incorporated by reference into the specification. The name of the text file containing the Sequence Listing is 44643662_ST25.txt. The text file is 75 KB, was created on Nov. 3, 2023, and is being submitted electronically via Patent Center.

### FIELD OF THE DISCLOSURE

The current disclosure provides methods and systems for gene-editing to increase functional factor VIII expression for the treatment of hemophilia A. The disclosure further provides nanoparticles to preferentially deliver gene-editing components to liver sinusoidal endothelial cells (LSEC) to correct mutant factor VIII genes.

### BACKGROUND OF THE DISCLOSURE

Hemophilia A is a common genetic bleeding disorder with an incidence of 1 in 5000 males worldwide. This genetic disease can result from various mutations within the coagulation Factor VIII (F8) gene. Factor VIII is an essential component of the blood clotting cascade. Clinically, hemophilia A can be classified based on relative Factor VIII activity in the patient's plasma as mild (5-30% activity; 60% of patients), moderate (2-5% activity; 14% of patients), or severe (<1% activity; 26% of patients).

Currently, there is no cure for hemophilia A. Standard therapy includes the administration of recombinant Factor VIII, but this approach is limited by cost, the requirement for frequent injections, and the formation of Factor VIII-inactivating antibodies in the subject which reduce the effectiveness of therapy. Therefore, a clear need still exists for alternative treatments for hemophilia A.

### SUMMARY OF THE DISCLOSURE

The current disclosure provides methods and systems for gene-editing to increase functional factor VIII expression for the treatment of hemophilia A. The disclosure further provides nanoparticles to preferentially deliver gene-editing components to liver sinusoidal endothelial cells (LSEC) to correct mutant factor VIII genes.

In particular embodiments, gene-editing components include a (1) guide RNA sequence and (2) nuclease and or nucleotide sequence encoding a nuclease. In particular embodiments, the guide RNA includes mF8 sgRNA as set forth in SEQ ID NO: 1. In particular embodiments, the guide RNA includes NSGHA sgRNA as set forth in SEQ ID NO: 2. In particular embodiments the nuclease includes Cas9.

In particular embodiments, the gene-editing components are associated with a nanoparticle. In particular embodiments, the nanoparticle is a chondroitin sulfate nanoparticle (ChS NP) or a lipid nanoparticle (LNP). In particular embodiments, the ChS NP includes chondroitin sulfate, oleylamine, and sorbitan monooleate. In particular embodiments, the LNP includes an ionic lipid, a helper lipid, cholesterol, a polyethylene glycol (PEG)-lipid conjugate, and an LSEC targeting agent.

### BRIEF DESCRIPTION OF THE SEVERAL VIEWS OF THE DRAWINGS

Some of the drawings submitted herein may be better understood in color. Applicant considers the color versions of the drawings as part of the original submission and reserves the right to present color images of the drawings in later proceedings.

FIG. 1. Depiction of blood clotting and blood clotting pathways. FIG. 1 shows a damaged blood vessel with associated bleeding. In individuals with functional blood clotting, the damage is repaired by blood clotting proteins and bleeding stops. In individuals with blood clotting disorders, however, the blood clotting proteins do not function and bleeding continues. There are at least 10 proteins that contribute to blood clotting: Factor XII, Factor XI, Factor IX, Factor VIII, Factor VII, Tissue Factor, Factor V, Factor X, Thrombin, and Fibrin. FVIII, encoded by the F8 gene, functions as a co-factor in the blood coagulation cascade. Defects in this F8 gene result in hemophilia A of varying degrees: severe hemophilia A: <1% of FVIII in the blood. 45% of severe hemophilia A is caused by an intron 22 inversion; 16% of severe hemophilia A is caused by small deletions/insertions; 3% of severe hemophilia A is caused by large deletions; 2% of severe hemophilia A is caused by an intron 1 inversion.

FIG. 2. CRISPR-Based Technologies to repair the mutant F8 gene. (Left panel): Insertion and deletion (Indel) mutation is generated from double strand break (DSB) followed by non-homologous end joining (NHEJ) repair. (Middle panel): A DSB is introduced into a mutant gene and a premature stop codon is repaired by Indel mutation. (Right panel): Precision repair. A DNA template with homology arms is introduced into a DSB so that a mutant region is fully repaired (e.g., by homology-mediated end joining (HMEJ)).

FIG. 3. Schematic of gene editing of the F8 gene in liver sinusoidal endothelial cells including location of sinusoidal endothelial cells and schematic of CRISPR/Cas9 acting on F8 genomic DNA.

FIG. 4. Sequences associated with immunodeficient mouse models: NOD.Cg-Prkdc^{scid} II2rg^{tm1Wjl}/SzJ (NSG) and relevant guide RNAs and amino acids, showing indel repair by CRISPR/Cas9. The exon 1 sequence of wild type mouse (WT mF8 exon 1) and NSG HemA mouse (mutant mF8 exon 1) are listed side by side and the deleted sequences of mutant mF8 are framed in black in wild type (WT) mF8 sequence. The sequence recognized by mF8 sgRNA is underlined. sgRNA sequences that target WT and mutant mF8 exon 1 are framed and labeled as mF8 sgRNA and NSGHA sgRNA, respectively. The premature stop codon resulted from frameshift are labeled by asterisk.

FIG. 5. In vitro gene editing by mF8 sgRNA. T7E1 assay of sgRNA and Cas9 protein transfected NIH3T3 cells. Grey arrow indicates the cutting band of mF8 sgRNA. Percentages of indels mutations were estimated by densitometer software. Fn1 stands for the fibronectin gene.

FIGS. 6A-6C. In vivo gene editing in mFVIII. (6A) Schematic in which NSG HemA mice undergo hydrodynamic injection of mF8 sgRNA or NSGHAsgRNA expression plasmid into the tail vein. (6B) Hydrodynamic injection of 100 μg NSGHA (n=4) and mF8 (n=2) sgRNA expression plasmid into the tail vein was performed in NSG HemA mice. Livers were harvested and genomic DNA were extracted 1 week after injection. Targeted region was analyzed by online sequence analysis tool. (6C) FVIII activity was measured for hydrodynamic injection of Lenti-Cas9 and lentivirus administrations of lentivurs/NSGHA sgRNA. Control mouse was administered PBS only.

FIGS. 7A-7E. (7A) Structure of endothelial cell-targeting chondroitin sulfate (ChS) nanoparticles (NP) encapsulating plasmid DNA (NP+pDNA). (7B) Synthesis methods for LSEC-targeting NP. Manufacturing components and steps include (1) DNA: p2X-GFP; (2) chondroitin sulfate nanoparticles; (3) Span80 (6.6 mg/ml), OA: (0.33 mg/ml) synthesized using a NanoAssemblr or nanoprecipitation (5) Amicon (10K) concentrate; and (6) Dialysis against water. (7C) Characterization of Chs-NP+pDNA by agarose gel electrophoresis. M, molecular weight marker; pDNA, plasmid DNA only control; NP: nanoparticle. The pDNA is efficiently encapsulated in the Chs-NP and retarded from migrating into the gel as shown in the gel retardation assay. The Chs-NP used in this assay is a nanoparticle with sorbitan monooleate (SP) as the targeting component. (7D) Characterization of size distribution and concentration of the Chs-NP; (7E) Significant transfection of HUVEC cells were observed at Day 1 using the synthesized Chs-NP. Different transfection media was used: Opti-Mem or GM (growth media). NC: negative control without NPs. (7F) NC: negative control without Chs-NP; PC opti-Mem: Chs-NP transfection with Opti-Mem as the media during transfection; PC GM: Chs-NP transfection with growth media (M) as the media during transfection. Both experiments showed significant transfection in HUVEC cells using Chs-NP encapsulating GFP plasmids.

FIGS. 8A-8C. In vitro and in vivo transfection efficiency of ChS-SP NP encapsulating GFP plasmids (GFP NP). (8A) HUVEC cells were transfected with buffer only (control), Lipo LTX and GFP plasmid, and GFP NP, respectively. Left: phase image of the cultured HUVEC cells. Right: fluorescence image of GFP gene expression in HUVEC cells. ChS-SP NP enhance the delivery of the GFP plasmid and achieves transfection in HUVEC cells. The transfection efficiency of ChS-SP NP encapsulating GFP plasmids (GFP NP) is almost as good as the positive control using commercial Lipo LTX transfecting agent. (8B) Flow cytometry analysis of GFP expression in HUVEC cells, top panel: control; middle panel: Lipo LTX +pGFP; bottom panel, GFP NP. (8C) Mice (n=5/group) were i.v. injected with ChS-SP NP encapsulating luciferase plasmid (Luc NP). One day following treatment, mouse livers were harvested to examine luciferase activity in liver lysate.

FIGS. 9A-9E. Encapsulation efficiency of Lipid Nanoparticle (LNP). The LNP is made differently than the ChS-SP NP. The LNP includes ionic lipids, helper lipids, cholesterol, and PEG-lipids. (9A) Agarose gel electrophoresis showed the encapsulation efficiency of nucleic acid in different (N/P ratio). Free pDNA were loaded as size control (Con). Marker (M). (9B) Agarose gel electrophoresis showed efficacy of Luciferase mRNA association with LNPs. Luciferase expression level in the treated (9C) HUVEC cells are presented as average luciferase signals +standard deviations in bioluminescence light units (BLU). Luciferase mRNA was formulated with KC2 formulation 1 (KC2-LNP-1), and 2 (KC2-LNP-2) and MC3 formulation B (MC3-LNP-B), respectively. (9D) 0.3 mg/kg of MC3 and KC2 Luc LNPs was administered to HemA mice intravenously. The control mice were treated with the same amount of mRNA only. The luciferase expression level was examined using in vivo imaging software (IVIS) system 4 hours after Luc LNPs injection. Data are presented as average±standard deviation. Statistical analyses were performed using one-way analysis of variance (ANOVA). **** p<0.0001. (9E) Schematic of an LNP.

FIG. 10. LSEC-targeting capacity of different MC3 GFP mRNA LNP formulations in vivo. Mice were intravenously injected with GFP mRNA encapsulated MC3 LNPs, modified cholesterol MC3 LNP (20α-OH MC3 LNP) and mannose-conjugated MC3 LNP (Man MC3 LNP), respectively at mRNA dose of 0.6-0.88 mg/kg. Mice livers were harvested 6 hours after injection. GFP (green) expression was analyzed. Liver sinusoidal endothelial cell marker: LYVE (red), Nuclear counterstain: DAPI (blue).

FIGS. 11A, 11B. In vivo gene editing by Cas9/sgRNA LNPs. (11A) NSG HemA mice were intravenously injected with Cas9/mF8 sgRNA LNPs and FVIII activities were evaluated by aPTT assay. (11B) Liver genomic DNA from Cas9/mF8 sgRNA LNPs treated NSG HemA mouse was analyzed by online sequence analysis tool.

FIG. 12. Sequences supporting the disclosure.

### DETAILED DESCRIPTION

Hemophilia A is a common genetic bleeding disorder with an incidence of 1 in 5000 males worldwide. Hemophilia A can result from various mutations within the coagulation Factor VIII (F8) gene. Factor VIII is an essential component of the clotting cascade. The protein circulates in the body in an inactive form that is attached to von Willebrand factor. In response to injury, Factor VIII is activated (Factor VIIIa) and separates from von Willebrand factor, then interacts with Factor IXa as part of the clotting cascade which leads to the formation of fibrin and stable clotting.

The Factor VIII gene located on the X chromosome is large and structurally complex, including 180 kb and 26 exons. The wild-type Factor VIII gene encodes two proteins. The first protein is the full-length Factor VIII protein, which is encoded by the 9030 bases found in exons 1 to 26, and has a circulating form containing 2332 amino acid residues. The second protein, referred to as Factor VIIIb, is encoded by 2598 bases in 5 exons present in the Factor VIII gene. The resulting protein includes 216 amino acids and has a presently unknown function. Hemophila A is associated with large deletions, insertions, inversions, and point mutations within the Factor VIII gene.

Clinically, hemophilia A can be classified based on relative Factor VIII activity in the patient's plasma as mild (5-30% activity; 60% of patients), moderate (2-5% activity; 14% of patients), or severe (<1% activity; 26% of patients). Currently, there is no cure for hemophilia A. Standard therapy includes the administration of recombinant Factor VIII, but this approach is limited by cost, the requirement for frequent injections, and the formation of Factor VIII-inactivating antibodies in the subject which reduce the effectiveness of therapy. Therefore, a clear need still exists for alternative treatments for hemophilia A.

The current disclosure provides in vivo gene repair strategies using nanoparticles that target endothelial cells and programmable nuclease systems to correct mutated Factor VIII (FVIII) genes and increase FVIII expression in hemophilia A patients. Specific sgRNAs are provided to correct small deletions/insertions together with CRISPR/Cas9 gene editing tools by indel repair or, for example, homologous recombination (HDR). Base editing can also be used to more efficiently correct specific base mutations to achieve precision repair. Delivery of Cas9 mRNA, sgRNAs and large fragment single strand oligonucleotides (ssODNs (a type of DNA repair template)) are used for correcting large deletions/inversions in FVIII gene via HDR. Therapeutic correction of the hemophilia A phenotype can be achieved using these strategies. These technologies serve as novel gene editing tools for personalized treatment of hemophilia A patients.

In particular embodiments, gene-editing components include (1) a guide RNA sequence and (2) a nuclease and or nucleotide sequence encoding a nuclease. In particular embodiments, the guide RNA includes mF8 sgRNA as set forth in SEQ ID NO: 1. In particular embodiments, the guide RNA includes NSGHA sgRNA as set forth in SEQ ID NO: 2. In particular embodiments the nuclease includes Cas9.

In particular embodiments, the gene-editing components are associated with a nanoparticle that preferentially delivers gene-editing components to LSEC. In particular embodiments, the nanoparticle is a chondroitin sulfate nanoparticle (ChS NP) or a lipid nanoparticle (LNP). In particular embodiments, the ChS NP includes chondroitin sulfate, oleylamine, and sorbitan monooleate as the LSEC targeting agent. In particular embodiments, the LNP includes an ionic lipid, a helper lipid, cholesterol, a PEG-lipid conjugate (referred to herein as a PEG-lipid), and a mannose or RGD peptide LSEC targeting agent.

Aspects of the current disclosure are now described in more supporting detail as follows: (i) Factor VIII and Mouse Models of Hemophilia A; (ii) Targeted Genetic Engineering; (iii) LSEC-Targeted Nanoparticles; (iv) Compositions for Administration; (v) Methods of Use; (vi) Exemplary Embodiments; (vii) Experimental Examples; and (viii) Closing Paragraphs. These headings are provided for organizational purposes only and do not limit the scope or interpretation of the disclosure.

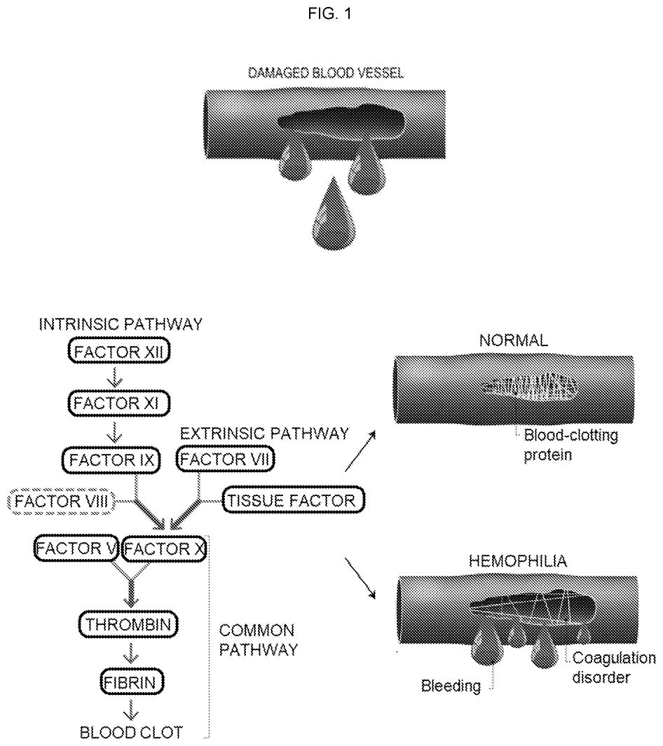

(i) Factor VIII and Mouse Models of Hemophilia A. The FVIII gene, located on the X chromosome, encodes a coagulation factor (Factor VIII) involved in the coagulation cascade that leads to clotting. Factor VIII is chiefly made by cells in the liver, and circulates in the bloodstream in an inactive form, bound to von Willebrand factor. Upon injury, FVIII is activated. The activated protein (FVIIIa) interacts with coagulation factor IX, leading to clotting (see FIG. 1).

Mutations in the FVIII gene cause hemophilia A. For example, mutations in FVIII can lead to the production of an abnormally functioning FVIII protein or a reduced or absent amount of circulating FVIII protein, leading to the reduction of or absence of the ability to clot in response to injury. Over 2100 mutations in the gene have been identified, including point mutations, deletions, and insertion, and ninety-eight percent of patients with a diagnosis of hemophilia A are found to have a mutation in the FVIII gene (i.e., intron 1 and 22 inversions, point mutations, insertions, and deletions). One of the most common mutations includes inversion of intron 22, which leads to a severe type of hemophilia A.

In humans, the Factor VIII gene, identified by NCBI as Gene ID No. 2157, is located from base pair 154,835,788 to base pair 155,026,934 on the X chromosome. In particular embodiments, Factor VIII in humans includes the sequence as set forth in SEQ ID NO: 13. In particular embodiments, Factor VIII in mouse includes the sequence as set forth in SEQ ID NO: 14.

In particular embodiments, a hemophilia A mouse model includes a five nucleotide deletion in exon 1 of the FVIII gene, resulting in a frameshift mutation that leads to a premature stop codon. (Chen et al., 2020, Mol Ther Nucleic Acids, 20:534-544. doi:10.1016/j.omtn.2020.03.015, PMC7178004, PMID:32330871). No inhibitor response is produced when NSG HemA mice are subjected to repeated infusions of FVIII protein or FVIII gene therapy. Thus, gene correction in this specific HemA mouse model can be easily evaluated over time without the complication of anti-FVIII inhibitor formation. The NSG HemA mouse model serves as a representative model for correction of small region gene mutations including missense, nonsense and frameshift mutations found in HemA patients. Use of this model is described in the Experimental Examples section of this disclosure.

In particular embodiments, a hemophilia A mouse model (B6; 129S-F8^{tm1Kaz/J}) includes a replacement of intron 16 resulting in disruption of FVIII expression. (Bi et al., 1996, Blood, 88(9):3446-50, PMID:8896409). There is no detectable plasma FVIII protein and FVIII activity (<1%) in these mice that are widely used for the evaluating efficacy of FVIII gene therapy. (Yen et al., 2016, Thromb J, 14(Suppl 1):22. doi: 10.1186/s12959-016-0106-0, PMC5056469, PMID:2776604). However, in vivo gene correction in MFVIII-16 HemA mice remains difficult since it is laborious to insert large fragment of DNA without assistance of viral vectors. (Chen et al., 2019, Sci Rep, 9(1): 16838. doi: 10.1038/s41598-019-53198-y, PMC6856096, PMID:31727959). This model can be used as representative models for repair of large deletions/insertions/inversions in FVIII gene. Use of this model is also described in the Experimental Examples section of this disclosure.

(ii) Targeted Genetic Engineering. Within the teachings of the current disclosure, any gene editing system capable of precise sequence targeting and modification can be used. These systems typically include a targeting element for precise targeting and a cutting element for cutting the targeted genetic site. Guide RNA is one example of a targeting element while various nucleases provide examples of cutting elements. Targeting elements and cutting elements can be separate molecules or linked, for example, by a nanoparticle. Alternatively, a targeting element and a cutting element can be linked together into one dual purpose molecule. Different gene editing systems can adopt different components and configurations while maintaining the ability to precisely target, cut, and modify selected genomic sites.

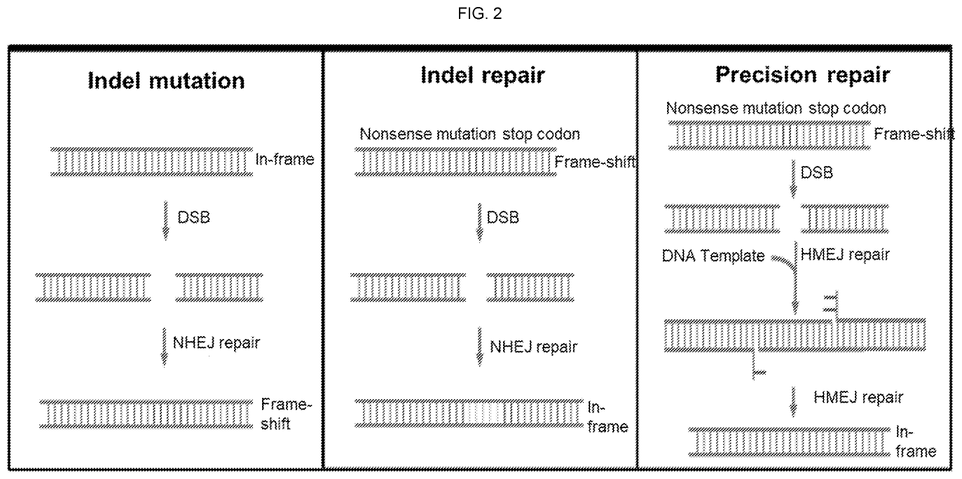

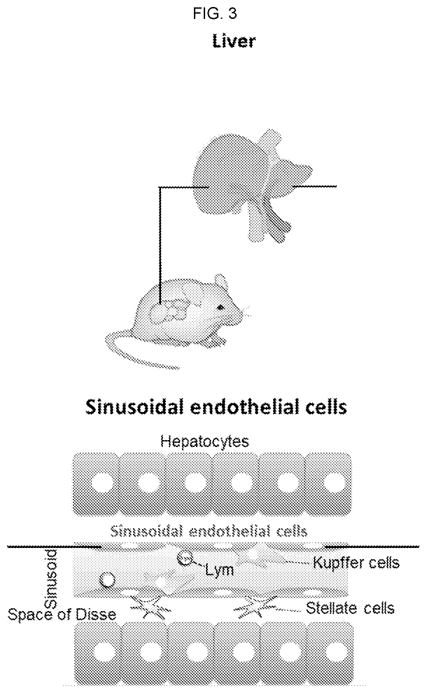

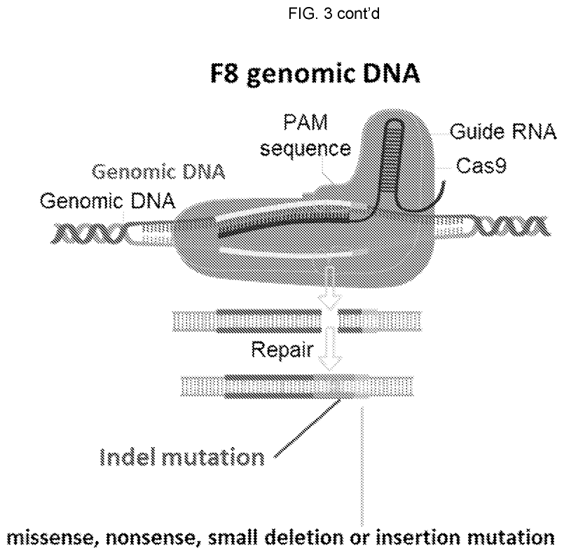

Engineered guide RNA associated with nucleases which target specific DNA sequences predictably generate DNA double strand breaks (DSB) at the targeted sequence. Use of gene editing systems to induce DSB can provide promising therapies when removal or silencing of a problematic gene (e.g., generating a loss-of-function mutation or creating an indel mutation or repair) is needed. Thus, gene-editing systems can be engineered to create a DSB at a desired target in a genome of a cell, and harness the cell's endogenous mechanisms to repair the induced break by non-homologous end joining (NHEJ) (see, e.g., the left and middle panel of FIG. 2 depicting DSB/NHEJ mediated indel mutation and indel repair).

When insertion of a therapeutic nucleic acid sequence is intended, the systems can also include a homology-directed repair template (“DNA Template” of FIG. 2, right panel; also referred to herein as a DNA repair template) which can include homology arms associated with the therapeutic nucleic acid sequence. In this instance, engineered guide RNA is again associated with nucleases which target specific DNA sequences predictably generating DSB at the targeted sequence. Following creation of a DSB at the desired target in the genome of a cell, the cell's endogenous mechanisms to repair the induced break is harnessed by homology repair, such as HDR) homology-mediated end joining (HMEJ), homology-independent targeted integration (HITI)-associated microhomology-mediated end joining (MMEJ), or HITI-associated non-homologous end joining (HITI-NHEJ) generally depending on the length of homology arms (e.g., as used herein HDR occurs if a region of homology is >75 bp and HITI occurs if a region of homology is <75 bp).

For gene addition or correction, homology-directed repair (HDR) of a DSB can be used. In this situation, gene-editing components generally include the engineered guide RNA and nuclease, and a homology-directed repair template with homology to the target DSB locus flanking a therapeutic gene.

HDR refers to DNA repair that takes place in cells, for example, during repair of double-stranded and single-stranded breaks in DNA. HDR requires nucleotide sequence homology between sequences of an HDR template and the target nucleic acid to repair the sequence where the break occurred in the target nucleic acid. In particular embodiments, the HDR template includes a non-homologous donor polynucleotide (donor sequence) flanked by two regions of homology (i.e., the homology arms), such that HDR between the target nucleic acid region and the two flanking homology arms results in insertion of the non-homologous donor polynucleotide at the target region. In particular embodiments, the homology arms will have at least 50% sequence identity to a genomic sequence with which recombination is desired. In particular embodiments, at least 60%, at least 70%, at least 80%, at least 90%, at least 95%, at least 98%, at least 99%, or at least 99.9% sequence identity is present between a homology arm and a target nucleic acid sequence. In particular embodiments, each homology arm can be 50 base pairs (bp), 100 bp, 125 bp, 150 bp, 175 bp, 200 bp, 225 bp, 250 bp, 275 bp, 300 bp, 325 bp, 350 bp, 375 bp, 400 bp, 425 bp, 450 bp, 475 bp, 500 bp, 525 bp, 550 bp, 575 bp, 600 bp, 625 bp, 650 bp, 675 bp, 700 bp, 725 bp, 750 bp, 775 bp, 800 bp, 825 bp, 850 bp, 875 bp, 900 bp, 925 bp, 950 bp, 975 bp, 1000 bp, 1250 pb, 1500 bp, or longer. In particular embodiments, the length of each homology arm can depend on the size of the donor polynucleotide and the target nucleic acid.

A DNA repair template includes a polynucleotide that can be directed to and inserted into a target site of interest to modify a target nucleic acid (e.g., in a genome). In particular embodiments, a DNA repair template is used as a template to copy the donor polynucleotide sequences into the target site of interest. Repair of the break in the target nucleic acid sequence can result in the transfer of genetic information (i.e., polynucleotide sequences) from the DNA repair template at the site or in close proximity of the break in the target nucleic acid sequence. Accordingly, new genetic information (i.e., polynucleotide sequences) may be inserted or copied at a target nucleic acid site. HDR may result in alteration of the target nucleic acid sequence (e.g., insertion, deletion, mutation) if the DNA repair template sequence differs from the target nucleic acid sequence. The DNA repair template may contain at least one or more single base changes, insertions, deletions, inversions or rearrangements with respect to the genomic sequence, so long as sufficient homology is present between sequences of the homology arms and the target nucleic acid sequence to support HDR. In particular embodiments, an entire DNA repair template, a portion of the DNA repair template, or a copy of the donor polynucleotide is integrated at the site of the target nucleic acid sequence. In particular embodiments, insertion or copying of the DNA repair template leads to correction of endogenous genes.

In particular embodiments, HMEJ-based repair is used to increase precision gene editing in non-dividing cells. The DNA template in HMEJ is similar to HDR, but the homologous regions are flanked by sgRNA targeting sites. Compared to MMEJ, HMEJ harbors longer homology arms to achieve higher gene repairing efficiency. In particular embodiments, the DNA repair template is excised from a plasmid or AAV vector.

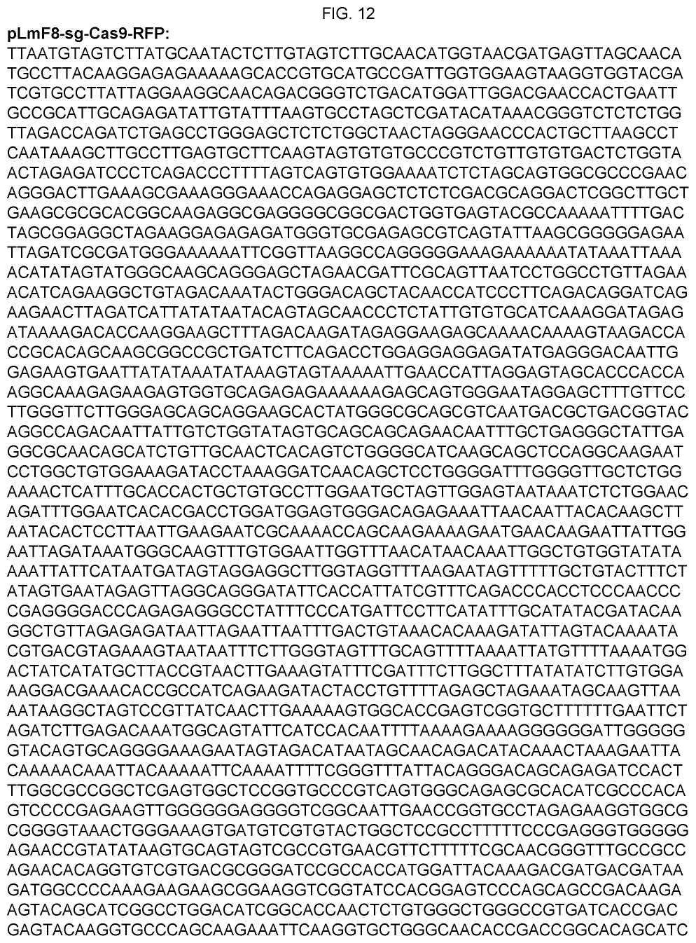

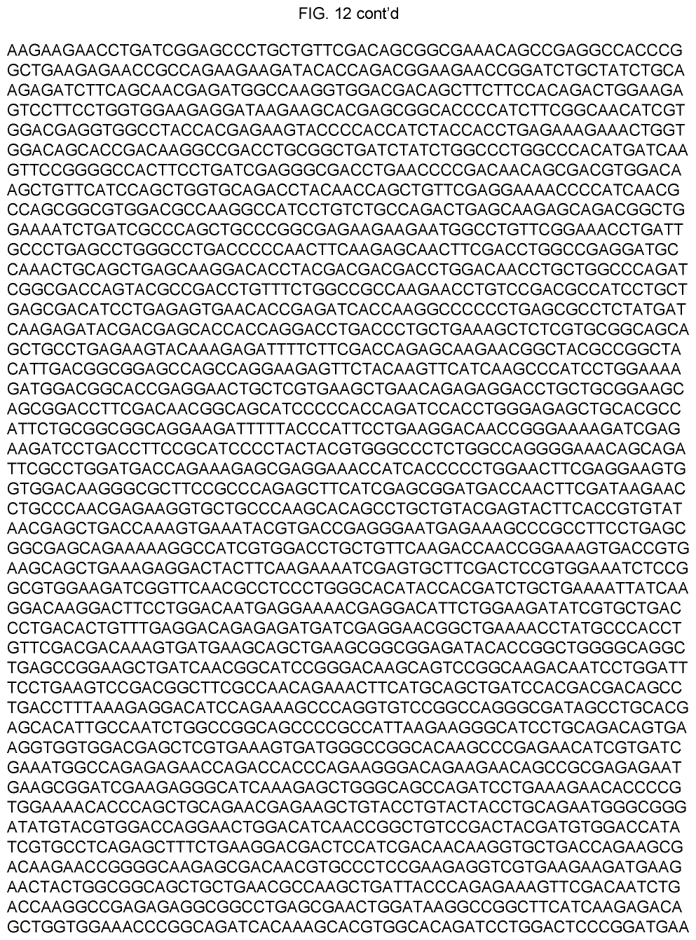

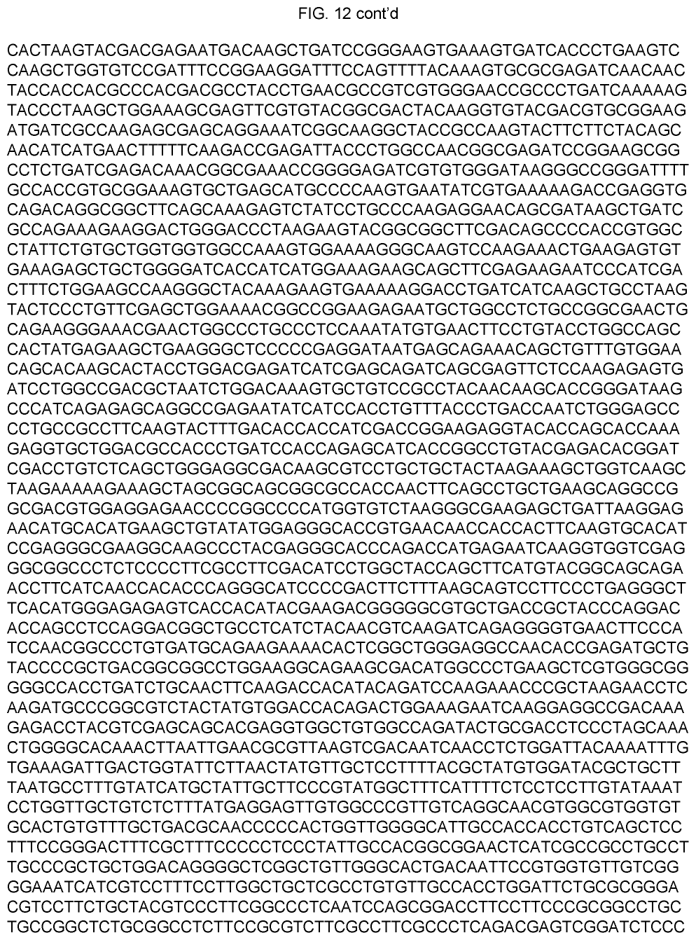

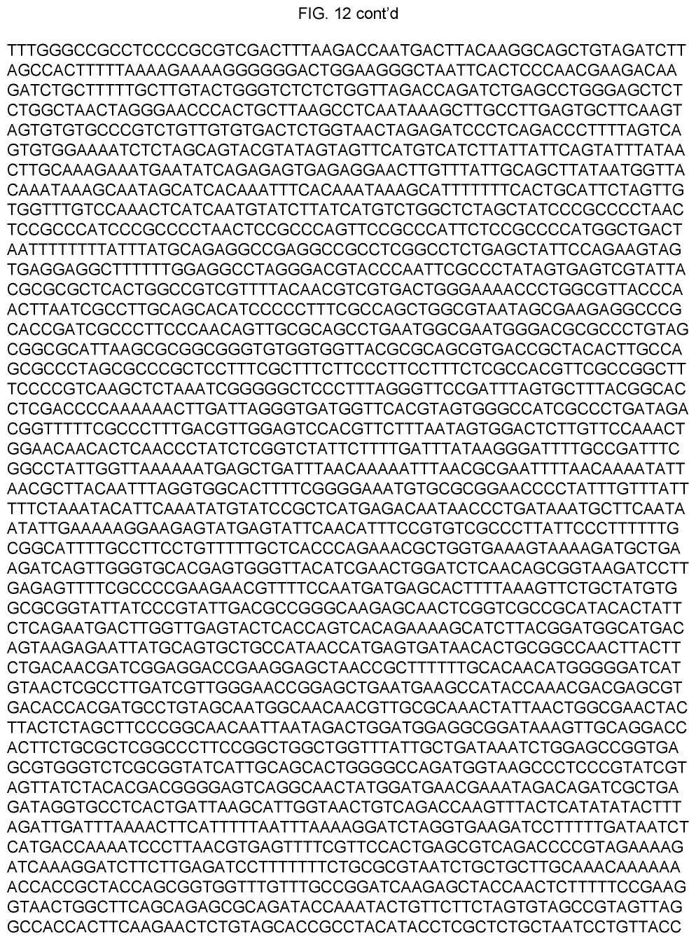

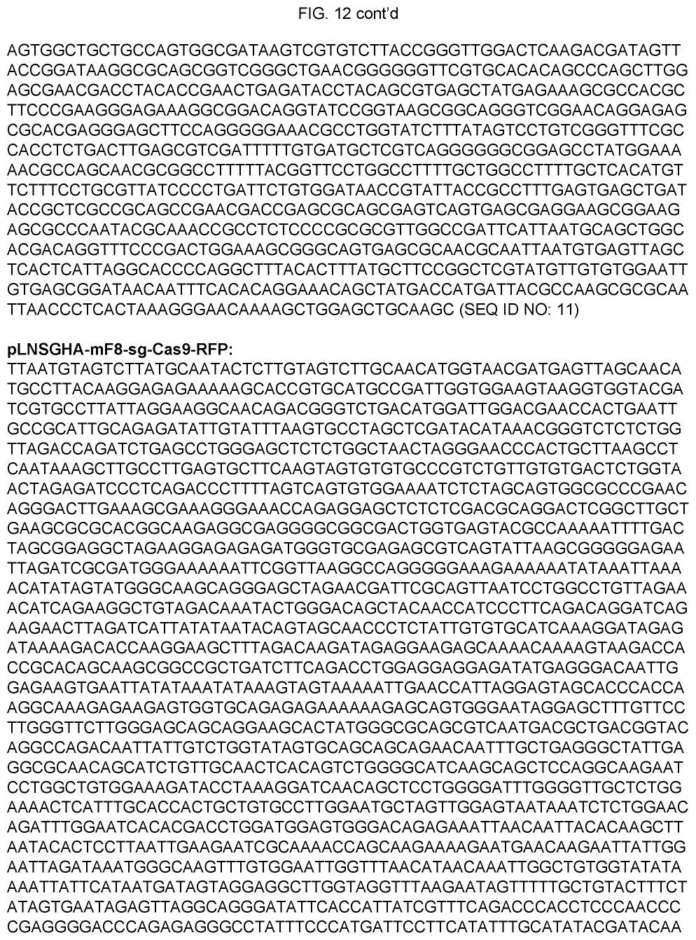

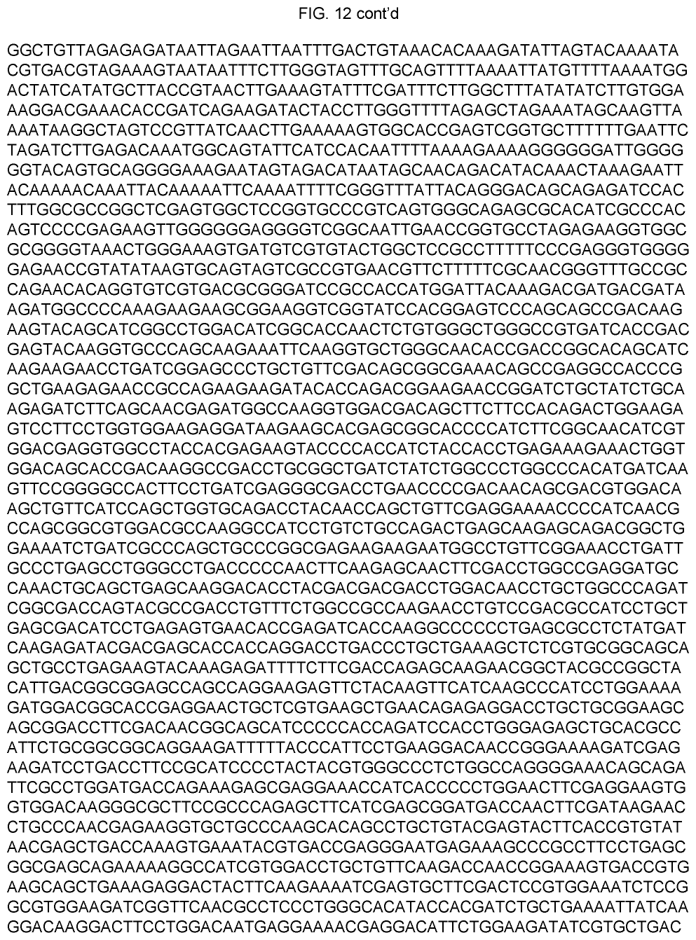

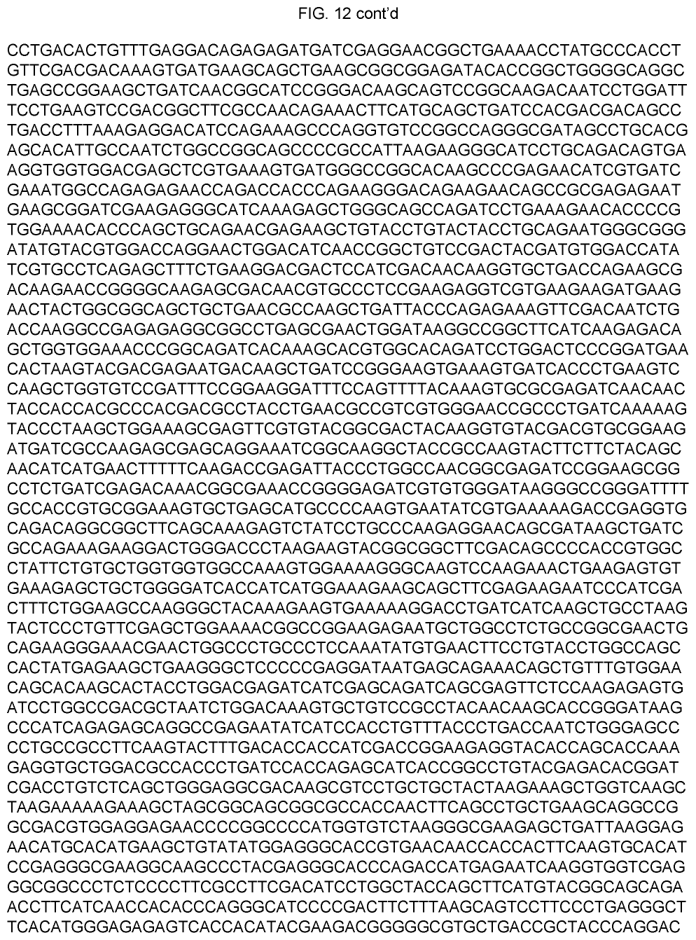

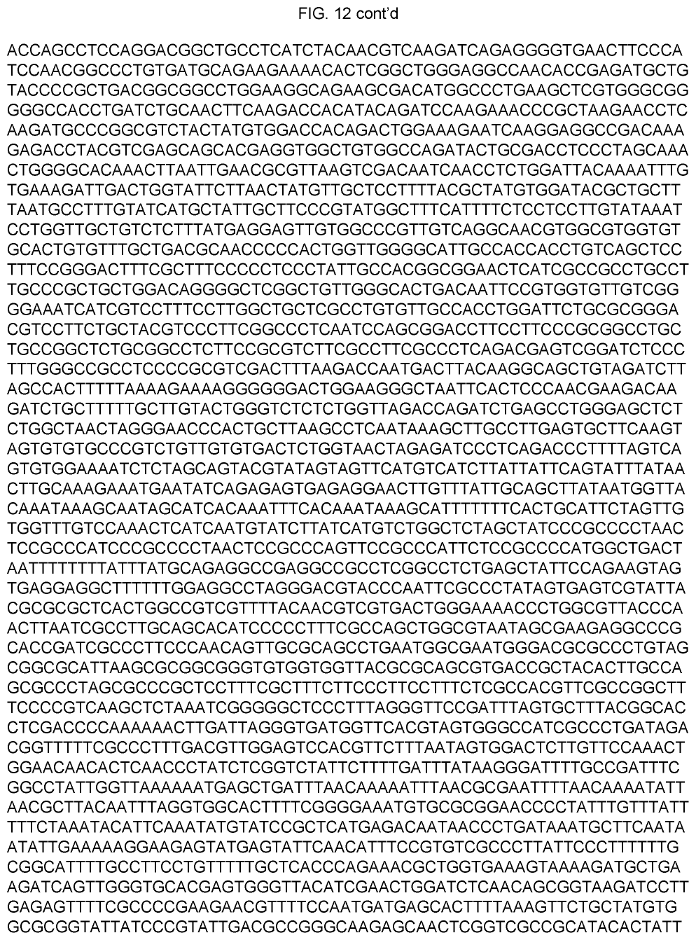

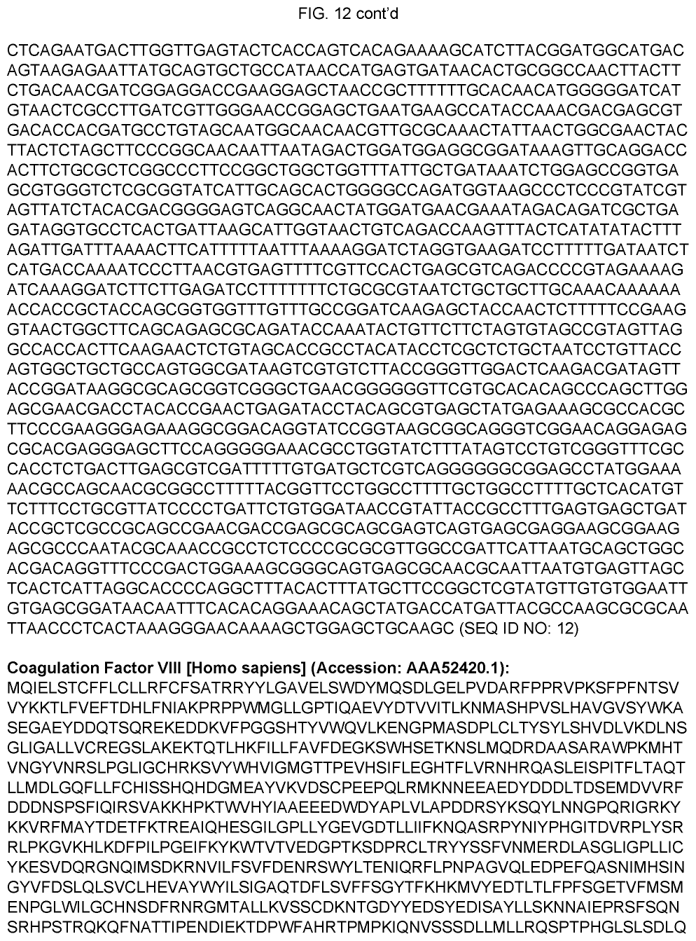

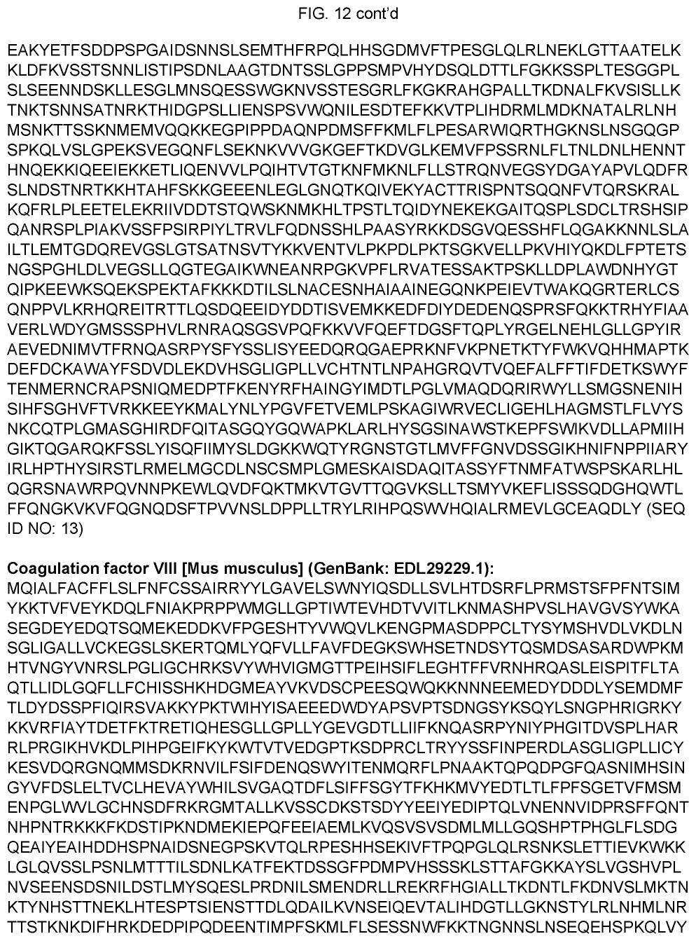

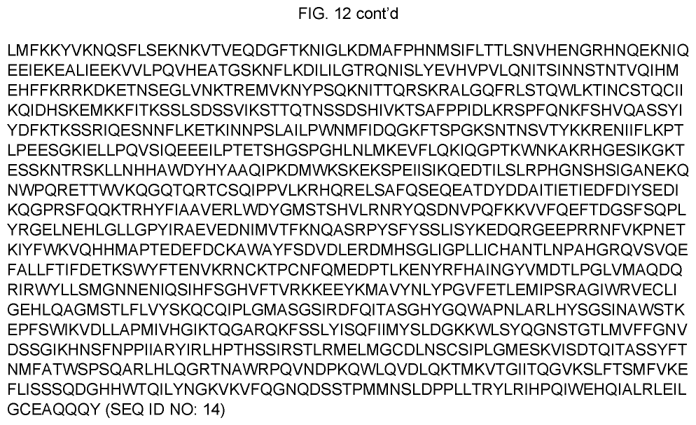

In particular embodiments, the donor polynucleotide can include a gene of interest. In particular embodiments, a gene of interest includes a polynucleotide that encodes functional Factor VIII. In particular embodiments, the gene of interest can include a polynucleotide sequence to modify a regulatory sequence of a gene, to introduce a regulatory sequence to a gene (e.g., a promoter, an enhancer, an internal ribosome entry sequence, a start codon, a stop codon, a localization signal, or polyadenylation signal), or to modify a nucleic acid sequence (e.g., introduce a mutation). Gene sequences encoding Factor VIII can be readily identified by those of ordinary skill in the art. Human and murine FVIII protein sequences are provided in FIG. 12 as SEQ ID NO: 13 AND 14, respectively.

Particular embodiments use the CRISPR gene editing system to provide functional Factor VIII expression.

Particular embodiments combine CRISPR RNA (crRNA) and trans-activating CRISPR RNA (tracrRNA) into a guide RNA (gRNA) or synthetic single guide RNA (sgRNA). In particular embodiments, a gRNA or sgRNA are the RNA molecules used to specify a particular target area for cleavage by a nuclease. In particular embodiments, gRNA includes two parts: crRNA, a nucleotide sequence (e.g., 17-20 nucleotides) complementary to the target DNA, and a tracrRNA sequence, which serves as a binding scaffold for the Cas nuclease. When the crRNA and tracrRNA elements are combined into a single RNA molecule, the molecule is referred to as sgRNA, though gRNA and sgRNA are often used interchangeably. In particular embodiments, gRNA includes sgRNA. For certain gene editing systems, the target sequence may be adjacent to a PAM (e.g., 5′-20nt target-NGG-3′) or can include a PAM. In particular embodiments, guide RNA (gRNA) includes a target site adjacent to the PAM targeted by the genome editing complex. The gRNA can include at least the 16, 17, 18, 19, 20, 21, or 22 nucleotides adjacent to the PAM.

In particular embodiments, the gRNA is mF8 sgRNA or NSGHA sgRNA. In particular embodiments, mF8 sgRNA includes the sequence: GCCATCAGAAGATACTACCT (SEQ ID NO: 1). In particular embodiments NSGHA sgRNA includes the sequence: ATCAGAAGATACTACCTTGG (SEQ ID NO: 2).

Exemplary CRISPR-Cas nucleases include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof.

A single Cas enzyme can be programmed by a gRNA molecule to site-specifically cleave a specific target nucleic acid. Cas9 is an exemplary Type II CRISPR Cas protein. Cas9 includes two distinct endonuclease domains (HNH and RuvC/RNase H-like domains), one for each strand of the target nucleic acid. RuvC and HNH together produce DSBs; separately each domain can produce single-stranded breaks. Base-pairing between the gRNA and target nucleic acid causes DSBs due to the endonuclease activity of Cas9. Binding specificity is determined by both gRNA-target nucleic acid base pairing and the PAM juxtaposed to the DNA complementary region. In particular embodiments, the CRISPR system only requires a minimal set of two molecules—the Cas protein and the gRNA.

A large number of Cas9 orthologs are known in the art (Fonfara et al. Nucleic Acids Research (2014) 42:2577-2590; Chylinski et al. Nucleic Acids Research (2014) 42:6091-6105; Esvelt et al. Nature Methods (2013) 10:1116-1121). A number of orthogonal Cas9 proteins have been identified including Cas9 proteins from Neisseria meningitidis, Streptococcus thermophilusand Staphylococcus aureus. Other Class 2 Cas proteins that can be used include Cas 12a (Cpf1), Cas13a (C2c2), and Cas13B (C2c6).

In particular embodiments, polynucleotide sequences encoding mutant forms of Cas9 nuclease can be used in genetic constructs of the disclosure. For example, a Sniper Cas9, a variant of Cas9 with optimized specificity (minimal off-target effects) and retained on-target activity can be used (Lee et al. J Vis Exp. 2019 Feb 26;(144); Lee et al. Nat Commun. 2018 Aug 6;9(1):3048; WO 2017/217768). As another example, a mutant Cas9 nuclease containing a D10A amino acid substitution can be used. This mutant Cas9 has lost double-stranded nuclease activity present in the wild type Cas9 but retains partial function as a single-stranded nickase. This mutant Cas9 generates a break in the complementary strand of DNA rather than both strands. This allows repair of the DNA template using a high-fidelity pathway rather than non-homologous end joining (NHEJ). The higher fidelity pathway prevents formation of insertions/deletions at the targeted locus while maintaining ability to undergo homologous recombination (Cong et al. Science (2013) 339(6121):819-823). Paired nicking has been shown to reduce off-target activity by 50- to 1,500-fold in cell lines (Ran et al. Cell (2013) 154(6): 1380-1389).

In particular embodiments, a Cas protein can be fused to a heterologous polypeptide that provides for subcellular localization. Such heterologous peptides include, for example, a nuclear localization signal (NLS) such as the SV40 NLS for targeting to the nucleus (e.g., Lange et al. (2007) J. Biol. Chem. 282:5101-5105). Such subcellular localization signals can be located at the N-terminus, the C-terminus, or anywhere within the Cas protein. An NLS can include a stretch of basic amino acids and can be a monopartite sequence or a bipartite sequence.

In particular embodiments, a Cas protein can also include a heterologous polypeptide for ease of tracking or purification, such as a fluorescent protein, a purification tag, or an epitope tag. Examples of tags include green fluorescent protein (GFP), glutathione-S-transferase (GST), myc, Flag, hemagglutinin (HA), Nus, Softag 1, Softag 3, Strep, polyhistidine, biotin carboxyl carrier protein (BCCP), maltose binding protein (MBP), and calmodulin.

The Cpf1 nuclease particularly can provide added flexibility in target site selection by means of a short, three base pair recognition sequence (TTN), known as the protospacer-adjacent motif or PAM. Cpf1′s cut site is at least 18bp away from the PAM sequence, thus the enzyme can repeatedly cut a specified locus after indel (insertion and deletion) formation, increasing the efficiency of HDR.

Additional information regarding CRISPR-Cas systems and components thereof are described in U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233, 8,999,641, and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473, WO2015/089486, WO2016/205711, WO2017/106657, WO2017/127807, and applications related thereto.

Teachings of the disclosure in relation to CRISPR can be applied to other gene editing systems that similarly utilize nucleases.

Particular embodiments utilize zinc finger nucleases (ZFNs) as gene editing agents. ZFNs are a class of site-specific nucleases engineered to bind and cleave DNA at specific positions. ZFNs are used to introduce DSBs at a specific site in a DNA sequence which enables the ZFNs to target unique sequences within a genome in a variety of different cells. Moreover, subsequent to double-stranded breakage, HDR or non-homologous end joining (NHEJ) takes place to repair the DSB, thus enabling genome editing.

ZFNs are synthesized by fusing a zinc finger DNA-binding domain to a DNA cleavage domain. The DNA-binding domain includes three to six zinc finger proteins which are similar to those found in transcription factors. The DNA cleavage domain includes the catalytic domain of, for example, Fokl endonuclease. The Fokl domain functions as a dimer requiring two constructs with unique DNA binding domains for sites on either side of the target site cleavage sequence. The Fokl cleavage domain cleaves within a five or six base pair spacer sequence separating the two inverted half-sites.

For additional information regarding ZFNs and ZFNs useful within the teachings of the current disclosure, see, e.g., U.S. Pat. Nos. 6,534,261; 6,607,882; 6,746,838; 6,794,136; 6,824,978; 6,866,997; 6,933, 113; 6,979,539; 7,013,219; 7,030,215; 7,220,719; 7,241,573; 7,241,574; 7,585,849; 7,595,376; 6,903,185; 6,479,626; and U.S. Application Publication Nos. 2003/0232410 and 2009/0203140 as well as Gaj et al., Nat Methods, 2012, 9(8): 805-7; Ramirez et al., Nucl Acids Res, 2012, 40(12):5560-8; Kim et al., Genome Res, 2012, 22(7): 1327-33; Urnov et al., Nature Reviews Genetics, 2010, 11 :636-646; Miller, et al. Nature biotechnology 25, 778-785 (2007); Bibikova, et al. Science 300, 764 (2003); Bibikova, et al. Genetics 161, 1169-1175 (2002); Wolfe, et al. Annual review of biophysics and biomolecular structure 29, 183-212 (2000); Kim, et al. Proceedings of the National Academy of Sciences of the United States of America 93, 1156-1160 (1996); and Miller, et al. The EMBO journal 4, 1609-1614 (1985).

Particular embodiments can use transcription activator like effector nucleases (TALENs) as gene editing agents. TALENs refer to fusion proteins including a transcription activator-like effector (TALE) DNA binding protein and a DNA cleavage domain. TALENs are used to edit genes and genomes by inducing DSBs in the DNA, which induce repair mechanisms in cells. Generally, two TALENs must bind and flank each side of the target DNA site for the DNA cleavage domain to dimerize and induce a DSB. The DSB is repaired in the cell by NHEJ or HDR if an exogenous double-stranded donor DNA fragment is present.

As indicated, TALENs have been engineered to bind a target sequence of, for example, an endogenous genome, and cut DNA at the location of the target sequence. The TALEs of TALENs are DNA binding proteins secreted by Xanthomonas bacteria. The DNA binding domain of TALEs include a highly conserved 33 or 34 amino acid repeat, with divergent residues at the 12^{th} and 13^{th} positions of each repeat. These two positions, referred to as the Repeat Variable Diresidue (RVD), show a strong correlation with specific nucleotide recognition. Accordingly, targeting specificity can be improved by changing the amino acids in the RVD and incorporating nonconventional RVD amino acids.

The present disclosure can utilize base editing systems, for example those that utilize a deaminase. Deamination of a nucleotide can cause changes in the sequence of a nucleic acid. Deamination of adenosine (A) results in an A-T to G-C transition. Deamination of cytosine (C) results in a C-G to T-A transition. Collectively, cytosine and adenosine deamination can be used to cause transitions from A to G, T to C, C to T, or G to A.

Examples of cytosine deaminase enzymes (CBEs) include APOBEC1, APOBEC3A, APOBEC3G, evoAPOBEC, BE4-YE1, CDA1, and AID. APOBEC1. Examples of adenosine base editors (ABEs) include a mutant TadA adenosine deaminases (TadA*) that accepts DNA as its substrate.

Particular base editing systems include a deaminase associated with a DNA binding domain such as a catalytically impaired nuclease domain. The DNA binding domain can localize the deaminase to a target nucleic acid in which one or more nucleotides are deaminated by the deaminase. Catalytically impaired nuclease domains are engineered from reference nuclease domain sequences but have a reduced or no ability to cause DSBs as compared to the reference (e.g., a wild-type) sequence.

Base editing systems can include a DNA glycosylase inhibitor that serves to override natural DNA repair mechanisms that might otherwise repair the intended base editing. A DNA glycosylase inhibitor can be a uracil DNA glycosylase inhibitor protein (UGI). One exemplary UGI is described in Wang et al. (Gene 99:31-37, 1991).

Exemplary base editing enzymes are described in e.g., Komor 2016 Nature 533: 420-424; Rees 2017 Nat. Commun. 8: 15790), Koblan 2018 Nat. Biotechnol 36(9): 843-846; Komor 2017 Sci. Adv. 3(8): eaao4774), Kim 2017 Nat. Biotechnol. 35: 475-480), Li 2018 Nat. Biotechnol. 36: 324-327)), Nishida 2016 Science 353(6305): aaf8729)), Nishimasu 2018 Science 361(6408): 1259-1262)), Hu 2018 Nature 556: 57-63)), Gehrke 2018 Nat. Biotechnol. 36(10): 977-982)), Wang 2018 Nat. Biotechnol. 36: 946-949)), Jiang 2018 Cell Res. 28(8): 855-861)), Rees 2018 Nat. Rev Genet. 19(12): 770-788 and Kantor 2020 Int. J. Mol. Sci. 21(17): 6240.

Dual base editors can edit both adenine and cytosine. (see, e.g., Sakata 2020 Nature Biotechnology, 38(7), 865-869; Grünewald 2020 Nat. Biotechnol. 38:861-864), and Zhang 2020 Nat. Biotechnol. 38:856-860).

In certain examples, a genetic construct of the disclosure includes elements to transcribe gRNA, express nuclease protein, and provide for expression of functional Factor VIII.

The term “genetic construct” refers to a polynucleotide vehicle to introduce genetic material into a cell. In particular embodiments, the term genetic construct includes plasmids and vectors. Plasmids can be linear or circular. In particular embodiments, a genetic construct of the disclosure is circular and is linearized through action of the gene-editing components encoded on the genetic construct. Genetic constructs can include, for example, an origin of replication, a multicloning site, and/or a selectable marker. An expression genetic construct typically includes an expression cassette. The term “expression cassette” includes a polynucleotide construct that is generated recombinantly or synthetically and includes regulatory sequences operably linked to a selected polynucleotide to facilitate expression of the selected polynucleotide in a host cell. For example, the regulatory sequences can facilitate transcription of the selected polynucleotide in a host cell, or transcription and translation of the selected polynucleotide in a host cell.

The terms “nucleic acid”, “nucleotide sequence”, and “polynucleotide” are interchangeable. All refer to a polymeric form of nucleotides. The nucleotides may be deoxyribonucleotides (DNA) or ribonucleotides (RNA), or analogs thereof, and they may be of any length. Polynucleotides may perform any function and may have any secondary structure and three-dimensional structure. The terms include known analogs of natural nucleotides and nucleotides that are modified in the base, sugar and/or phosphate moieties. Analogs of a particular nucleotide have the same base-pairing specificity (e.g., an analog of A base pairs with T). A polynucleotide may include one modified nucleotide or multiple modified nucleotides. Examples of modified nucleotides include methylated nucleotides and nucleotide analogs. Nucleotide structure may be modified before or after a polymer is assembled. Following polymerization, polynucleotides may be additionally modified by, for example, conjugation with a labeling component or target-binding component. A nucleotide sequence may incorporate non-nucleotide components. The terms also include nucleic acids including modified backbone residues or linkages, that (i) are synthetic, naturally occurring, and non-naturally occurring, and (ii) have similar binding properties as a reference polynucleotide (e.g., DNA or RNA). Examples of such analogs include phosphorothioates, phosphoramidates, methyl phosphonates, chiral-methyl phosphonates, 2-O-methyl ribonucleotides, peptide-nucleic acids (PNAs), and morpholino structures.

The term “complementarity” refers to the ability of a nucleic acid sequence to form hydrogen bond(s) with another nucleic acid sequence (e.g., through traditional Watson-Crick base pairing). A percent complementarity indicates the percentage of residues in a nucleic acid molecule that can form hydrogen bonds with a second nucleic acid sequence. When two polynucleotide sequences have 100% complementarity, the two sequences are perfectly complementary, i.e., all of a first polynucleotide's contiguous residues hydrogen bond with the same number of contiguous residues in a second polynucleotide.

In particular embodiments, the term “gene” refers to a nucleic acid sequence (used interchangeably with polynucleotide or nucleotide sequence) that encodes a protein (e.g., a nuclease protein or a therapeutic protein), a negative selection marker, a selectable marker, or gRNA, as described herein. This definition includes various sequence polymorphisms, mutations, and/or sequence variants wherein such alterations do not substantially affect the function of the encoded protein or gRNA. The nucleic acid sequences can include both the full-length nucleic acid sequences as well as non-full-length sequences derived from a full-length protein. The sequences can also include degenerate codons of the native sequence or sequences that may be introduced to provide codon preference in a specific cell type. In particular embodiments, the term “gene” may include not only coding sequences but also regulatory regions such as promoters, enhancers, 5′ UTR, 3′UTR, termination regions, and non-coding regions. The term further can include all introns and other DNA sequences spliced from an mRNA transcript, along with variants resulting from alternative splice sites. Gene sequences encoding a molecule can be DNA or RNA that directs the expression of the molecule. These nucleic acid sequences may be a DNA strand sequence that is transcribed into RNA or an RNA sequence that is translated into protein.

“Encoding” refers to the property of specific sequences of nucleotides in a gene, such as a complementary DNA (cDNA), or a messenger RNA (mRNA), to serve as templates for synthesis of other macromolecules such as a defined sequence of amino acids or a functional polynucleotide (e.g., gRNA, siRNA). In particular embodiments, a gene encodes or codes for a protein if transcription and translation of mRNA corresponding to that gene produces the protein in a cell or other biological system. A “gene sequence encoding a protein” includes all nucleotide sequences that are degenerate versions of each other and that code for the same amino acid sequence or amino acid sequences of substantially similar form and function. In particular embodiments, a gene encodes or codes for a functional polynucleotide when transcription of the gene produces the functional polynucleotide. In particular embodiments, the functional polynucleotide includes gRNA.

The terms “regulatory sequences”, “regulatory elements”, and “control elements” are interchangeable and refer to polynucleotide sequences that are upstream (5′ non-coding sequences), within, or downstream (3′ non-translated sequences) of a polynucleotide sequence to be transcribed or expressed. In particular embodiments, upstream and downstream relate to the 5′ to 3′ direction, respectively, in which RNA transcription takes place. In particular embodiments, upstream is toward the 5′ end of a nucleic acid and downstream is toward the 3′ end of a nucleic acid. Regulatory sequences influence, for example, the timing of transcription, amount or level of transcription, RNA processing or stability, and/or translation of a polynucleotide sequence. Regulatory sequences may include activator binding sequences, enhancers, introns, polyadenylation recognition sequences, promoters, repressor binding sequences, stem-loop structures, translational initiation sequences, translation leader sequences, transcription termination sequences, translation termination sequences, primer binding sites, and the like. The term “operably linked” refers to polynucleotide sequences or amino acid sequences placed into a functional relationship with one another. For instance, a promoter or enhancer is operably linked to a coding sequence or to a non-coding sequence (e.g., gRNA) if it regulates, or contributes to the modulation of, the transcription of the coding or non-coding sequence. In particular embodiments, regulatory sequences operably linked to a coding sequence or non-coding sequence are typically contiguous to the coding sequence or non-coding sequence. However, enhancers can function when separated from a promoter by up to several kilobases or more. Accordingly, some polynucleotide elements may be operably linked but not contiguous.

(iii) LSEC-Targeted Nanoparticles. Exemplary LSEC-targeted nanoparticles can include an LSEC targeting ligand on the surface of the nanoparticle wherein the LSEC targeting ligand results in preferential delivery of the nanoparticle to the LSEC. In certain examples, preferential delivery means that LSEC cells uptake at least 10% more, at least 20% more, at least 30% more, at least 40% more, at least 50% more, at least 60% more, at least 70% more, at least 90% more or at least 100% more of administered nanoparticles than a reference cell type. In certain examples, the reference cell type is hepatocytes. In other examples, the reference cell type is a non-liver cell, such as a lung cell or spleen cell. Nanoparticles disclosed herein preferentially delivery gene-editing components (e.g., gRNA, nuclease, and/or DNA repair template) to LSEC.

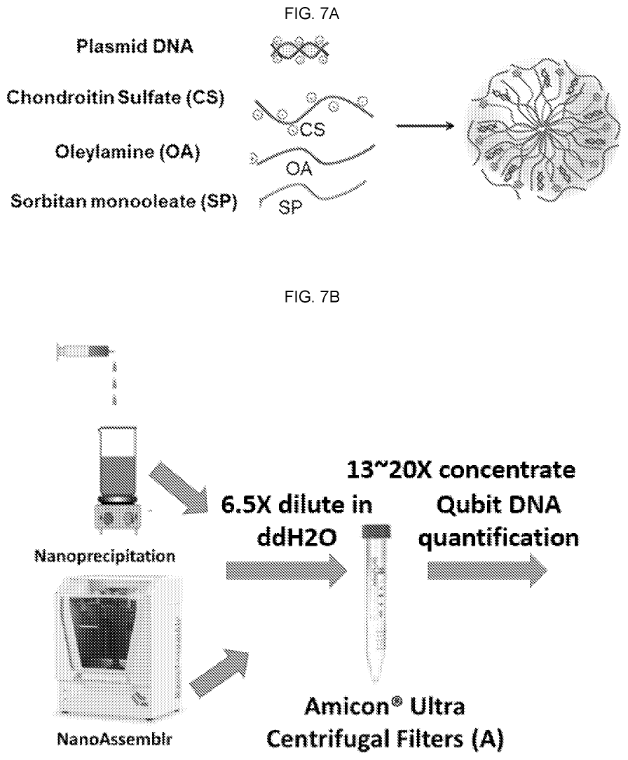

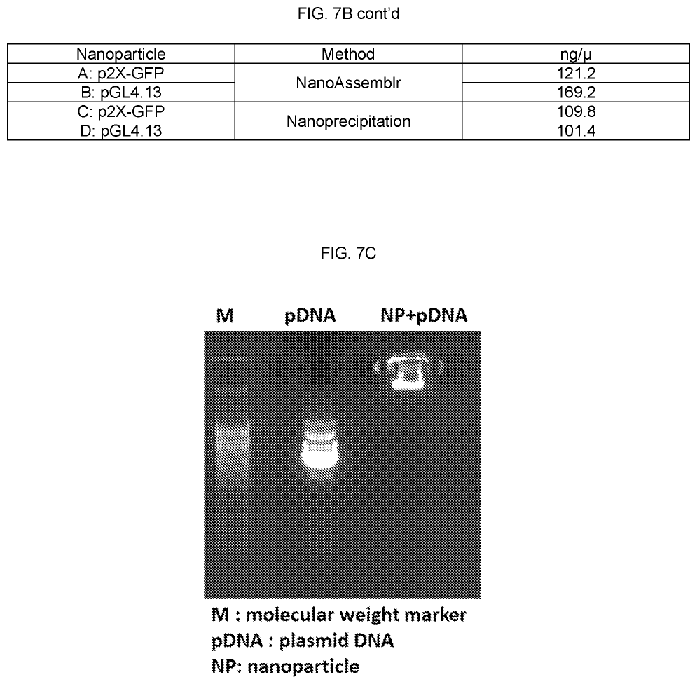

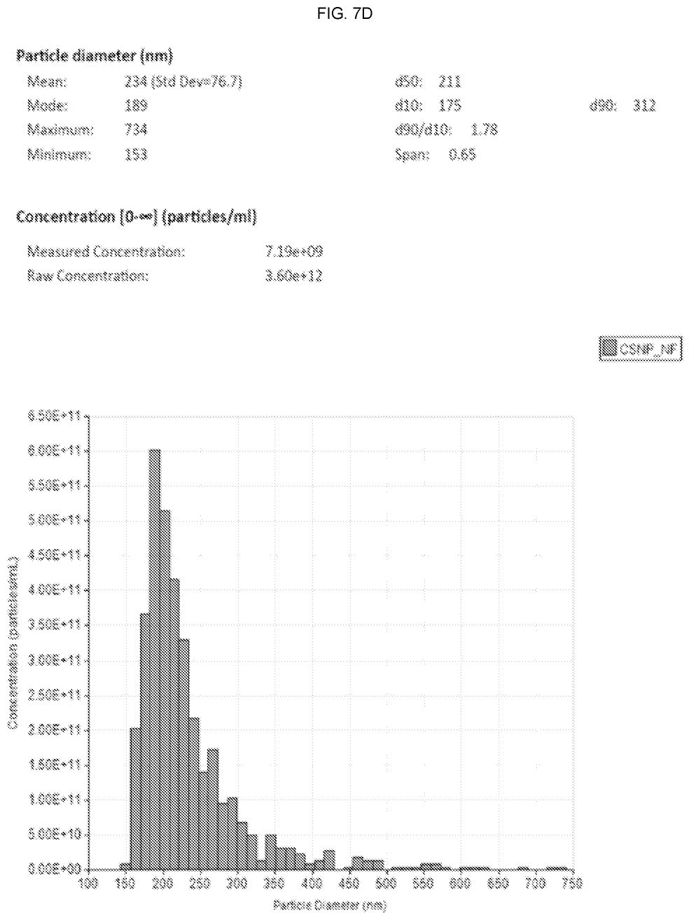

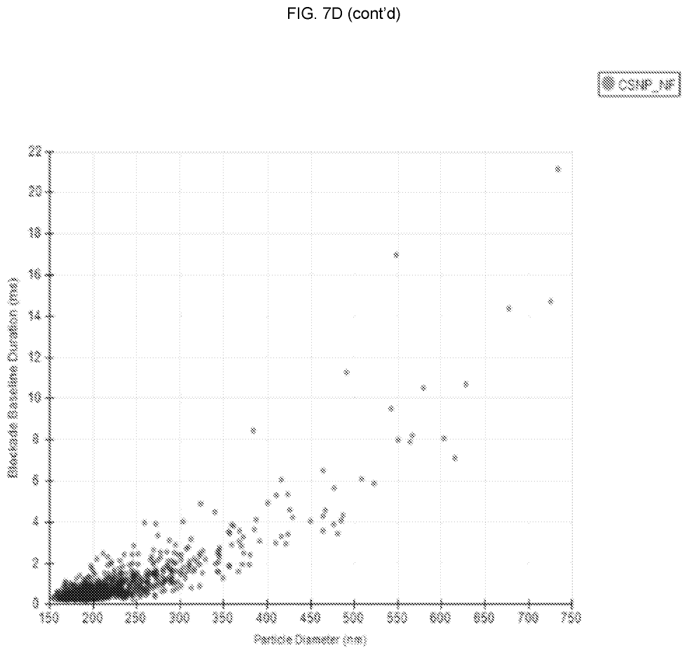

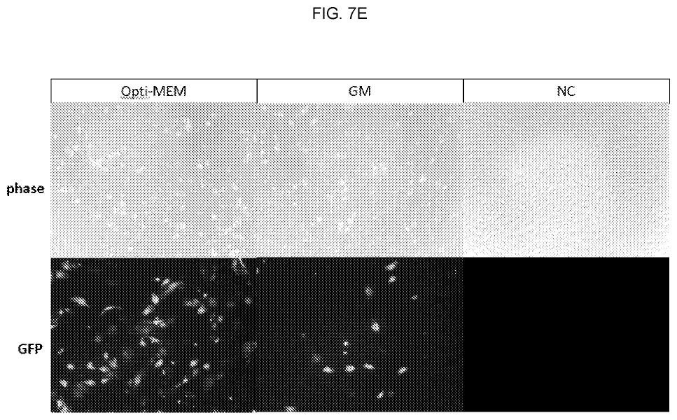

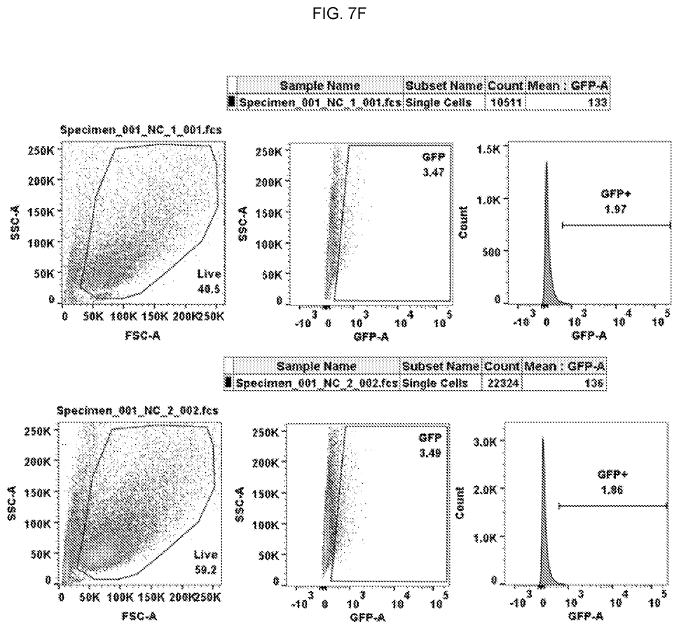

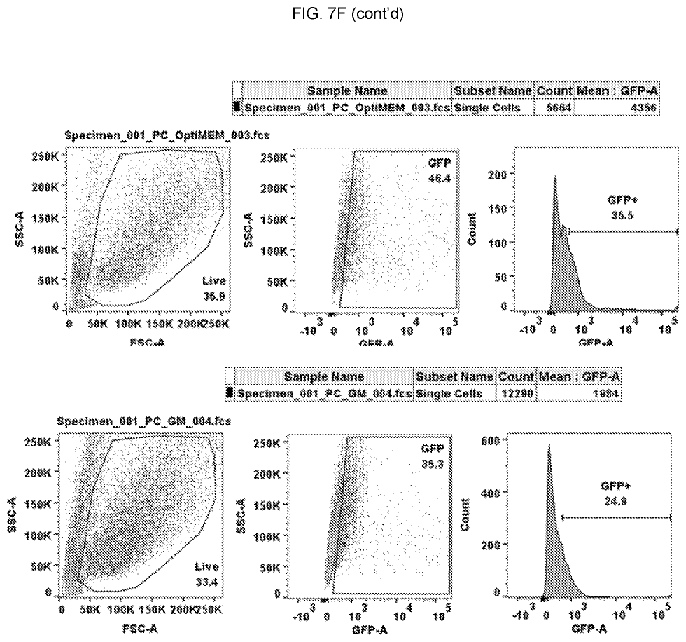

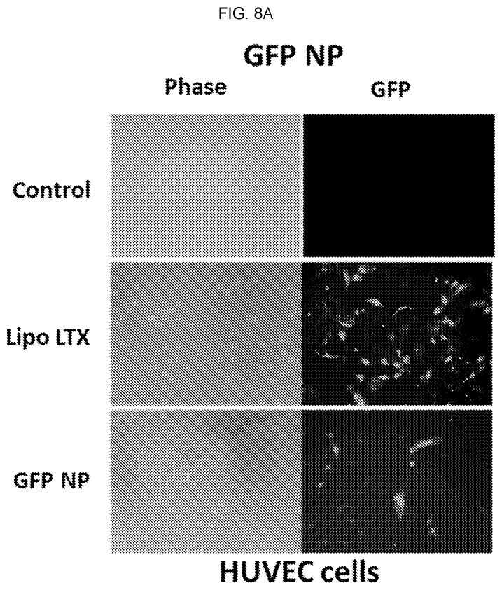

In particular embodiments, a chondroitin sulfate nanoparticle (ChS NP) includes chondroitin sulfate (ChS), oleylamime (OA), and sorbitan monooleate (SP) as an LSEC targeting agent. See FIG. 7A for the structure of the ChS NP and location of plasmid DNA with in the ChS NP.

In some embodiments, gene-editing components are encapsulated within lipid nanoparticle (LNP). A typical LNP formulation is composed of pH-responsive lipids or cationic lipids bearing tertiary or quaternary amines to encapsulate the polyanionic mRNA. Helper lipids are usually neutral lipids like dioleoyl phosphatidyl ethanolamine (DOPE), 1,2-Distearoyl-sn-glycero-3-phosphoethanolamine (DSPE), or 1,2-distearoyl-sn-glycero-3-phosphocholine (DSPC), and/or sterol lipids such as cholesterol to stabilize the lipid bilayer of the LNP and to enhance mRNA delivery efficiency. Polyethylene glycol (PEG)-lipid is used to improve the colloidal stability in a biological environment such as in blood circulation by reducing the specific absorption of plasma proteins and forming a hydration layer over the nanoparticles. The morphology of lipid nanoparticles is not like a traditional liposome, characterized by a lipid bilayer surrounding an aqueous core. LNPs possess an electron-dense core where the cationic/ionizable lipids are organized into inverted micelles around the encapsulated mRNA molecules. In particular embodiments, an LNP includes an ionic lipid, a helper lipid, a cholesterol, and a PEG-lipid.

Particular embodiments of nanoparticles disclosed herein include:

| Ionic lipid | Helper lipid | Cholesterol | PEG-lipid |
|---|---|---|---|
| 30% | 1.25-10% | 18.5-48.5% | 0.75-6% |
| 40% | | | |
| 50% | | | |
| 60% | | | |

Particular embodiments of nanoparticles disclosed herein include:

| Ionic lipid | Helper lipid | Cholesterol | PEG-lipid |
|---|---|---|---|
| 30% | 1.25% | 48.5% | 1.5% |
| | 2.5% | 38.5% | 3% |
| | 5% | 18.5% | 6% |
| | 10% | 28.5% | 0.75% |
| 40% | 1.25% | 38.5% | 0.75% |
| | 2.5% | 48.5% | 6% |
| | 5% | 28.5% | 3% |
| | 10% | 18.5% | 1.5% |
| 50% | 1.25% | 18.5% | 3% |
| | 2.5% | 28.5% | 1.5% |
| | 5% | 48.5% | 0.75% |
| | 10% | 38.5% | 6% |
| 60% | 1.25% | 28.5% | 6% |
| | 2.5% | 18.5% | 0.75% |
| | 5% | 38.5% | 1.5% |
| | 10% | 48.5% | 3% |

In particular embodiments, a lipid nanoparticle includes 30%-60% ionic lipid, 1.25%-10% helper lipid, 18.5%-48.5% cholesterol, and 0.75%-6% PEG-lipid. In particular embodiments, a lipid nanoparticle includes 30% ionic lipid, 1.25%-10% helper lipid, 18.5%-48.5% cholesterol, and 0.75%-6% PEG-lipid. In particular embodiments, a lipid nanoparticle includes 40% ionic lipid, 1.25%-10% helper lipid, 18.5%-48.5% cholesterol, and 0.75%-6% PEG-lipid. In particular embodiments, a lipid nanoparticle includes 50% ionic lipid, 1.25%-10% helper lipid, 18.5%-48.5% cholesterol, and 0.75%-6% PEG-lipid. In particular embodiments, a lipid nanoparticle includes 60% ionic lipid, 1.25%-10% helper lipid, 18.5%-48.5% cholesterol, and 0.75%-6% PEG-lipid.

In particular embodiments, the ionic lipid includes MC3, KC2, 7C1, or cKK-E12.

In particular embodiments, MC3 refers to DLIN-MC3-DMA: (6Z,9Z,28Z,31Z)-heptatriacont-6.9.28.31-tetraene-19-yl 4-(dimethylamino) butanoate:

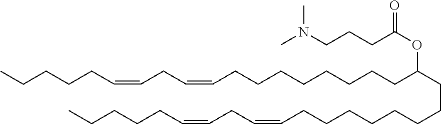 [SMILES: CCCCC/C=C\C/C=C\CCCCCCCCC(CCCCCCCC/C=C\C/C=C\CCCCC)OC(=O)CCCN(C)C]

In particular embodiments, KC2 refers to DLin-KC2-DMA: C43H79NO2

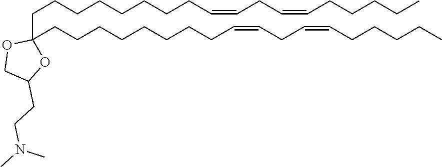 [SMILES: CCCCC/C=C\C/C=C\CCCCCCCCC1(CCCCCCCC/C=C\C/C=C\CCCCC)OCC(CCN(C)C)O1]

In particular embodiments, 7C1 is described in Nat. Nanotechnol. 2014 Aug; 9(8): 648-655. In particular embodiments, 7C1 refers to poly(ethyleneamine) with a C15 lipid:

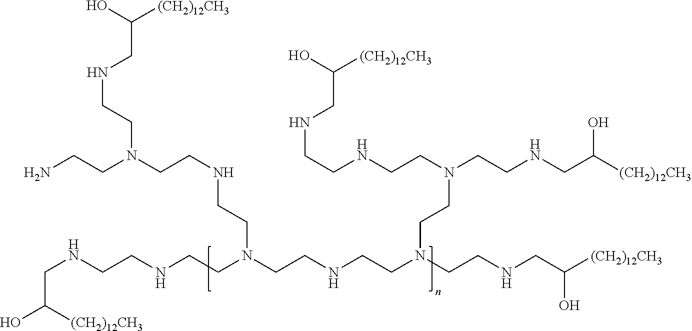

In particular embodiments, cKK-E12 has the molecular formula C₆₀H₁₂₀N₄O₆. In particular embodiments, cKK-E12 has the structure:

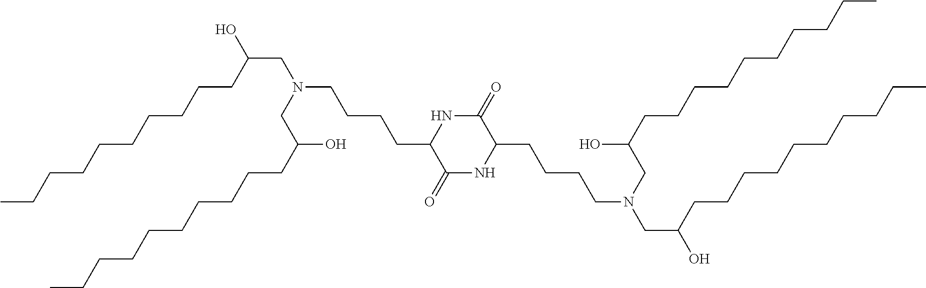

In particular embodiments, the ionic lipid is cationic lipid such as, e.g., 1,2-dilinoleyloxy-3-dimethylaminopropane (DLinDMA), 2,2-dilinoleyl-4-(2dimethylaminoethyl)-[1,3]-dioxolane (DLin-KC2-DMA), or heptatriaconta-6,9,28,31-tetraen-19-yl 4-(dimethylamino)butanoate (DLin-MC3-DMA).

Additional exemplary cationic lipids include N-[1-(2,3-dioleyloxy)propyl]-N,N,N-trimethylammonium chloride (DOTMA); N-[1-(2,3-dioleoyloxy)propyl]-N,N,N-trimethylammonium chloride (DOTAP); 1,2-dioleoyl-sn-glycero-3-ethylphosphocholine (DOEPC); 1,2-dilauroyl-sn-glycero-3-ethylphosphocholine (DLEPC); 1,2-dimyristoyl-sn-glycero-3-ethylphosphocholine (DMEPC); 1,2-dimyristoleoyl-sn-glycero-3-ethylphosphocholine (14:1), N1-[2-((1S)-1-[(3-aminopropyl)amino]-4-[di(3-amino-propyl)amino]butylcarboxamido)ethyl]-3,4-di[oleyloxy]-benzamide (MVL5); Dioctadecylamido-glycylspermine (DOGS); 3b-[N-(N′,N′-dimethylaminoethyl) carbamoyl]cholesterol (DC-Chol); Dioctadecyldimethylammonium Bromide (DDAB); a Saint lipid (e.g., SAINT-2, N-methyl-4-(dioleyl)methylpyridinium); 1,2-dimyristyloxypropyl-3-dimethylhydroxyethylammonium bromide (DMRIE); 1,2-dioleoyl-3-dimethyl-hydroxyethyl ammonium bromide (DORIE); 1,2-dioleoyloxypropyl-3-dimethylhydroxyethyl ammonium chloride (DORI); Di-alkylated Amino Acid (DILASup2/Sup) (e.g., C18:1-norArg-C16); Dioleyldimethylammonium chloride (DODAC); 1-palmitoyl-2-oleoyl-sn-glycero-3-ethylphosphocholine (POEPC); and 1,2-dimyristoleoyl-sn-glycero-3-ethylphosphocholine (MOEPC).

Additional examples include ioctadecyldimethylammonium bromide (DDAB), 1,2-Dioleoyloxy-3-dimethylaminopropane (DODAP), 1,2-Dioleyloxy-3-dimethylaminopropane (DODMA), Morpholinocholesterol (Mo-CHOL), (R)-5-(dimethylamino)pentane-1,2-diyl dioleate hydrochloride (DODAPen-C1), (R)-5-guanidinopentane-1,2-diyl dioleate hydrochloride (DOPen-G), (R)-N,N,N-trimethyl-4,5-bis(oleoyloxy)pentan-1-aminium chloride (DOTAPen). In certain embodiments, a lipid nanoparticle includes a combination or two or more cationic lipids.

In particular embodiments, a helper lipid includes dioleoylphosphatidylethanolamine (DOPE), 1,2-Distearoyl-sn-glycero-3-phosphoethanolamine (DSPE), or 1,2-distearoyl-sn-glycero-3-phosphocholine (DSPC). Additional helper lipids that can be used include 11,2-dipalmitoyl-sn-glycero-3-phosphocholine (DPPC); 1,2-dimyristoyl-sn-glycero-3-phosphocholine (DMPC); 1,2-dilauroyl-sn-glycero-3-phosphoethanolamine (DLPE); 1,2-dimyristoyl-sn-glycero-3-phosphoethanolamine (DMPE); and 1,2-diphytanoyl-sn-glycero-3-phosphoethanolamine (DPHyPE). In certain embodiments, a lipid nanoparticle includes a combination or two or more helper lipids.

In particular embodiments, cholesterol includes cholesterol or a similar structure. In particular embodiments, cholesterol includes 20α-Hydroxycholestrol.

Examples of cholesterol derivatives include polar analogues such as 5α-cholestanol, 5α-coprostanol, cholesteryl-(2′-hydroxy)-ethyl ether, cholesteryl-(4′-hydroxy)-butyl ether, and 6-ketocholestanol; non-polar analogues such as 5α-cholestane, cholestenone, 5α-cholestanone, 5α-cholestanone, and cholesteryl decanoate; and mixtures thereof. In particular embodiments, the cholesterol derivative is a polar analogue such as cholesteryl-(4′-hydroxy)-butyl ether.

In particular embodiments, the PEG-lipid includes DMG-PEG2000 (1,2-Dimyristoyl-rac-glycero-3-methoxypolyethylene glycol-2000). Additional polyethylenegycol-lipid conjugates (PEG-lipids) include N-(Carbonyl-methoxypolyethyleneglycoln)-1,2-dimyristoyl-sn-glycero-3-phosphoethanolamine (DMPE-PEGn where n is 350, 500, 750, 1000 or 2000), N-(Carbonyl-methoxypolyethyleneglycoln)-1,2-distearoyl-sn-glycero-3-phosphoethanolamine (DSPE-PEGn where n is 350, 500, 750, 1000 or 2000), DSPE-polyglycelin-cyclohexyl-carboxylic acid, DSPE-polyglycelin-2-methylglutar-carboxylic acid, polyethylene glycol-dimyristolglycerol (PEG-DMG), polyethylene glycol-distearoyl glycerol (PEG-DSG), and N-octanoyl-sphingosine-1-{(succinyl[methoxy(polyethylene glycol)2000]} (C8 PEG2000 Ceramide). In some variations of DMPE-PEGn where n is 350, 500, 750, 1000 or 2000, the PEG-lipid is N-(Carbonyl-methoxypolyethyleneglycol 2000)-1,2-dimyristoyl-sn-glycero-3-phosphoethanolamine (DMPE-PEG 2,000). In some variations of DSPE-PEGn where n is 350, 500, 750, 1000 or 2000, the PEG-lipid is N-(Carbonyl-methoxypolyethyleneglycol 2000)-1,2-distearoyl-sn-glycero-3-phosphoethanolamine (DSPE-PEG 2,000). In certain embodiments, a lipid nanoparticle includes a combination or two or more PEG-lipids.

In particular embodiments, the ionic lipid is MC3. In particular embodiments, the cholesterol is 20α-Hydroxycholestrol. In particular embodiments, the lipid nanoparticle is conjugated to mannose.

In certain examples, nanoparticles include MC3, helper lipid 1,2-dioleoyl-sn-glycero-3-phosphoethanolamine (DOPE), cholesterol (Chol) and 1,2-dimyristoyl-rac-glycero-3-methoxypolyethylene glycol-2000 (DMG-PEG2000) at a 50/10/39/1 mole ratio. These lipid components can be formulated with nucleic acid at a volume ratio of 1:3. In certain examples, the nitrogen and phosphate ratio (N/P) between ionic lipid and pDNA is between 6-8.

In certain examples, nanoparticles include KC2, helper lipid DOPE, Chol, and DMG-PEG2000 at a 50/10/39/1 mole ratio. These lipid components can be formulated with nucleic acid at a volume ratio of 1:3. In certain examples, the nitrogen and phosphate ratio (N/P) between ionic lipid and pDNA is between 6-8.

In some embodiments, gene-editing components, are coupled to a cell penetrating peptide or targeting ligand to facilitate LSEC uptake. Examples of cell penetrating peptides known in the art include poly-arginine (Jearawiriyapaisarn, et al. (2008) Mol Ther. 16:1624-9), TAT peptide from the HIV virus (Hudecz et al. (2005), Med. Res. Rev. 25: 679-736), MPG (Simeoni, et al. (2003) Nucleic Acids Res. 31:2717-2724), Pep-1 (Deshayes et al. (2004) Biochemistry 43: 7698-7706, and HSV-1 VP-22 (Deshayes et al. (2005) Cell Mol Life Sci. 62:1839-49). In an alternative embodiment, gene-editing components, are coupled covalently or non-covalently to an antibody that recognizes a specific cell-surface receptor expressed on LSEC such that the gene-editing components bind to and are internalized by the LSEC. Alternatively, gene-editing components can be coupled covalently or non-covalently to the natural ligand (or a portion of the natural ligand) for such a cell-surface receptor. (McCall, et al. (2014) Tissue Barriers. 2(4): e944449; Dinda, et al. (2013) Curr Pharm Biotechnol. 14:1264-74; Kang, et al. (2014) Curr Pharm Biotechnol. 15(3):220-30; Qian et al. (2014) Expert Opin Drug Metab Toxicol. 10(11):1491-508).

Other exemplary nanoparticle types include liposomes (microscopic vesicles including at least one concentric lipid bilayer surrounding an aqueous core), and liposomal nanoparticles (a liposome structure used to encapsulate another smaller nanoparticle within its core). Other polymer-based nanoparticles can also be used as well as porous nanoparticles constructed from any material capable of forming a porous network. Exemplary materials include metals, transition metals and metalloids (e.g., lithium, magnesium, zinc, aluminum and silica).

(iv) Compositions for Administration. Gene-editing components (e.g., sgRNA, nuclease, DNA repair templates, vectors) or gene-editing components incorporated within nanoparticles (all collectively “active ingredients”) can be formulated alone or in combination into compositions for administration to subjects. Salts and/or pro-drugs of active ingredients can also be used.

Exemplary generally used pharmaceutically acceptable carriers include any and all absorption delaying agents, antioxidants (e.g., ascorbic acid, methionine, vitamin E), binders, buffering agents, bulking agents or fillers, chelating agents (e.g., EDTA), coatings, disintegration agents, dispersion media, gels, isotonic agents, lubricants, preservatives, salts, solvents or co-solvents, stabilizers, surfactants, and/or delivery vehicles.

Exemplary antioxidants include ascorbic acid, methionine, and vitamin E.

Exemplary buffering agents include citrate buffers, succinate buffers, tartrate buffers, fumarate buffers, gluconate buffers, oxalate buffers, lactate buffers, acetate buffers, phosphate buffers, histidine buffers, and/or trimethylamine salts. An exemplary chelating agent is EDTA.

Exemplary isotonic agents include polyhydric sugar alcohols including trihydric or higher sugar alcohols, such as glycerin, erythritol, arabitol, xylitol, sorbitol, or mannitol.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Stabilizers refer to a broad category of excipients which can range in function from a bulking agent to an additive which solubilizes the active ingredient or helps to prevent denaturation or adherence to the container wall. Typical stabilizers can include polyhydric sugar alcohols; amino acids; organic sugars or sugar alcohols; sulfur-containing reducing agents; proteins such as human serum albumin, bovine serum albumin, gelatin or immunoglobulins; hydrophilic polymers such as polyvinylpyrrolidone; monosaccharides such as xylose, mannose, fructose and glucose; disaccharides; trisaccharides, and polysaccharides.

The formulations disclosed herein can be formulated for administration by, for example, injection. For injection, formulation can be formulated as aqueous solutions, such as in buffers including Hanks' solution, Ringer's solution, or physiological saline, or in culture media, such as Iscove's Modified Dulbecco's Medium (IMDM). Injectable formulations can be in lyophilized and/or powder form for constitution with a suitable vehicle, e.g., sterile pyrogen-free water, before use.

Compositions can be formulated as an aerosol. In particular embodiments, the aerosol is provided as part of an anhydrous, liquid or dry powder inhaler. Aerosol sprays from pressurized packs or nebulizers can also be used with a suitable propellant, e.g., dichlorodifluoromethane, trichlorofluoromethane, dichlorotetrafluoroethane, carbon dioxide or other suitable gas. In the case of a pressurized aerosol, a dosage unit may be determined by providing a valve to deliver a metered amount. Capsules and cartridges of gelatin for use in an inhaler or insufflator may also be formulated including a powder mix of active ingredients and a suitable powder base such as lactose or starch.

Compositions can also be formulated as depot preparations. Depot preparations can be formulated with suitable polymeric or hydrophobic materials (for example as an emulsion in an acceptable oil) or ion exchange resins, or as sparingly soluble derivatives, for example, as a sparingly soluble salt.

Any composition disclosed herein can advantageously include any other pharmaceutically acceptable carriers which include those that do not produce significantly adverse, allergic, or other untoward reactions that outweigh the benefit of administration. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety, and purity standards as required by U.S. FDA Office of Biological Standards and/or other relevant foreign regulatory agencies.

In particular embodiments, the compositions include active ingredients of at least 0.1% w/v or w/w of the composition; at least 1% w/v or w/w of composition; at least 10% w/v or w/w of composition; at least 20% w/v or w/w of composition; at least 30% w/v or w/w of composition; at least 40% w/v or w/w of composition; at least 50% w/v or w/w of composition; at least 60% w/v or w/w of composition; at least 70% w/v or w/w of composition; at least 80% w/v or w/w of composition; at least 90% w/v or w/w of composition; at least 95% w/v or w/w of composition; or at least 99% w/v or w/w of composition.

Compositions disclosed herein can be formulated for administration by, for example, injection, infusion, perfusion, or lavage. The compositions disclosed herein can further be formulated for intravenous, intradermal, intraarterial, intranodal, intralymphatic, intraperitoneal, intralesional, intraprostatic, intravaginal, intrarectal, intrathecal, intramuscular, intravesicular, and/or subcutaneous administration and more particularly by intravenous, intradermal, intraperitoneal, intramuscular, and/or subcutaneous injection.

(v) Methods of Use. Methods disclosed herein include treating subjects (e.g., humans, veterinary animals (dogs, cats, reptiles, birds) livestock (e.g., horses, cattle, goats, pigs, chickens) and research animals (e.g., monkeys, rats, mice, fish) with compositions disclosed herein. Treating subjects includes delivering therapeutically effective amounts. Therapeutically effective amounts include those that provide effective amounts and/or therapeutic treatments.

An “effective amount” is the amount of a composition necessary to result in a desired physiological change in the subject. Effective amounts are often administered for research purposes. Effective amounts disclosed herein can cause a statistically-significant effect in an animal model or in vitro assay relevant to the assessment of hemophilia A or in a clinical trial assessing the efficacy and safety of a hemophilia treatment.

A “therapeutic treatment” includes a treatment administered to a subject who displays symptoms or signs of hemophilia A and is administered to the subject for the purpose of diminishing or eliminating those signs or symptoms of hemophilia A. The therapeutic treatment can reduce, control, or eliminate the effects of hemophilia A.

Functions as an effective amount or therapeutic treatment are not mutually exclusive, and in particular embodiments, administered dosages may accomplish more than one treatment type.

In particular embodiments, therapeutically effective amounts provide reduction in symptoms of hemophilia A. A reduction in symptoms of hemophilia A can include an increase in functional Factor VIII expression and improved blood clotting following damage to a blood vessel.

In particular embodiments, the administration of a therapeutically effective amount results in an increase of functional Factor VIII in a subject's plasma of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or up to 100% compared to the level of functional Factor VIII in the subject's plasma prior to the administration.

In particular embodiments, the administration of a therapeutically effective amount results in a decrease in bleeding by a subject following injury to a blood vessel of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or up to 100% than the level observed prior to the administration following comparable damage to a blood vessel.

In certain examples, therapeutically effective amounts are confirmed by measuring and detecting an improvement in activated partial thromboplastin time (aPTT).

For administration, therapeutically effective amounts (also referred to herein as doses) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of hemophilia A, previous or concurrent therapeutic interventions, idiopathy of the subject and route of administration.

Useful doses can range from 0.1 to 5 μg/kg or from 0.5 to 1 μg/kg. In other non-limiting examples, a dose can include 1 μg/kg, 15 μg/kg, 30 μg/kg, 50 μg/kg, 55 μg/kg, 70 μg/kg, 90 μg/kg, 150 μg/kg, 350 μg/kg, 500 μg/kg, 750 μg/kg, 1000 μg/kg, 0.1 to 5 mg/kg or from 0.5 to 1 mg/kg. In other non-limiting examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly).

The pharmaceutical compositions described herein can be administered by, for example, injection, inhalation, infusion, perfusion, or lavage. Routes of administration can include intravenous, intradermal, intraparenteral, intranasal, intramuscular, and/or subcutaneous.

The Exemplary Embodiments and Example below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### (vi) Exemplary Embodiments

1. A method for genetically-modifying the Factor VIII gene in the genome of a mammalian cell, the method including delivering a therapeutically effective amount of an LSEC-targeted nanoparticle to a subject wherein the nanoparticle is associated with gene-editing components that repair a Factor VIII gene in the mammalian cell.
2. A method for genetically-modifying the Factor VIII gene in the genome of a mammalian cell, the method including delivering a therapeutically effective amount of gene-editing components that repair a Factor VIII gene in the mammalian cell.
3. The method of embodiment 1 or 2, wherein the gene-editing components include (1) mF8 single guide RNA (sgRNA) or NSGHA sgRNA and (2) a nuclease and/or a nucleotide sequence encoding the nuclease.
4. The method of embodiment 3, wherein the mF8 sgRNA includes the sequence as set forth in SEQ ID NO: 1 or a sequence having at least 97% or 98% sequence identity to the sequence as set forth in SEQ ID NO: 1.
5. The method of embodiment 3 or 4, wherein the NSGHA sgRNA includes the sequence as set forth in SEQ ID NO: 2 or a sequence having at least 97% or 98% sequence identity to the sequence as set forth in SEQ ID NO: 2.
6. The method of any of embodiments 1-5, wherein the gene-editing include the sequence as set forth in SEQ ID NO: 11 or SEQ ID NO: 12 or have at least 90% sequence identity to the sequence as set forth in SEQ ID NO: 11 or SEQ ID NO: 12.
7. The method of any of embodiments 1-6, wherein the therapeutically effective amount results in expression of functional Factor VIII.
8. The method of any of embodiments 1-7, wherein the mammalian cell is within a living subject.
9. The method of embodiment 8, wherein the expression of functional Factor VIII is at a level sufficient to ameliorate a symptom of hemophilia in the living subject.
10. The method of embodiment 8 or 9, wherein the living subject is a mouse or human.
11. The method of any of embodiments 1-10, wherein the gene-editing components include a nuclease.
12. The method of embodiment 11, wherein the nuclease includes Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, or Csf4.
13. The method of method of embodiment 11, wherein the nuclease is Cas9.
14. The method of any of embodiments 1-13, wherein the gene-editing components include a nucleotide sequence encoding the nuclease.
15. The method of embodiment 14, wherein the nucleotide sequence encoding the nuclease is in the form of plasmid DNA.
16. The method of any of embodiments 1-15, wherein the nanoparticle includes chondroitin sulfate, oleylamine, and sorbitan monooleate.
17. The method of any of embodiments 1-15, wherein the nanoparticle is a lipid nanoparticle.
18. The method of embodiment 17, wherein the lipid nanoparticle includes an ionic lipid, a helper lipid, cholesterol, and a PEG-lipid.
19. The method of embodiment 18, wherein the ionic lipid 7C1, MC3, KC2, or cKK-E12.
20. The method of embodiment 18 or 19, wherein the helper lipid includes dioleoylphosphatidylethanolamine (DOPE), 1,2-Distearoyl-sn-glycero-3-phosphoethanolamine (DSPE), or 1,2-distearoyl-sn-glycero-3-phosphocholine (DSPC).
21. The method of any of embodiments 18-20, wherein the cholesterol includes 20α-Hydroxycholestrol.
22. The method of any of embodiments 18-21, wherein the PEG-lipid includes DMG-PEG2000 (1,2-Dimyristoyl-rac-glycero-3-methoxypolyethylene glycol-2000).
23. The method of any of embodiments 1-22, wherein the nanoparticle includes mannose and/or arginylglycylaspartic acid (RGD) peptide.
24. The method of embodiment 23, wherein the mannose is conjugated to the PEG-lipid.
25. The method of embodiment 23, wherein the RGD peptide is conjugated to the helper lipid.
26. The method of any of embodiments 17-25, wherein the lipid nanoparticle includes MC3, 20α-Hydroxycholestrol, DSPE, DMG-PEG2000, and RGD peptide.
27. The method of any of embodiments 17-25, wherein the lipid nanoparticle includes MC3, 20α-Hydroxycholestrol, DSPE, DMG-PEG2000, and mannose.
28. The method of any of embodiments 1-27, wherein the gene-editing components include a DNA repair template.
29. The method of embodiment 28, wherein the DNA repair template encodes a functional Factor VIII protein and includes homology arms homologous to a region within 154,835,788 to 155,026,934 on the X chromosome.
30. The method of embodiment 28 or 29, wherein the DNA repair template is encoded by a viral vector.
31. The method of any of embodiments 28-30, wherein the DNA repair template is within a viral vector.
32. The method of any of embodiments 28-30, wherein the viral vector is an adeno associated viral vector.
33. The method of any of embodiments 1-32, wherein the administering increases the level of expression of functional Factor VIII in the mammalian cell.
34. The method of any of embodiments 8-33, wherein the administering increases the clotting rate by at least 10%, at least 20%, at least 30%, at least 40%, at least 50% as compared to the clotting rate in an untreated hemophilia subject with comparable damage to blood vessel.
35. The method of any of embodiments 8-34, wherein the administering is through intravenous, intradermal, intraarterial, intranodal, intravesicular, intrathecal, intraperitoneal, intraparenteral, intranasal, intralesional, intramuscular, oral, inhaled, subcutaneous, or sublingual administration.
36. The method of any of embodiments 8-34, wherein the administering is through intravenous administration.
37. The method of any of embodiments 1-36, wherein the administering includes administering multiple doses.
38. A nanoparticle associated with gene-editing components that result in repair of a Factor VIII gene in a mammalian cell, wherein the repair results in functional Factor VIII expression by the mammalian cell.
39. The nanoparticle of embodiment 38, including an mF8 sgRNA or an NSGHA sgRNA.
40. The nanoparticle of embodiment 39, wherein the mF8 sgRNA includes the sequence as set forth in SEQ ID NO: 1 or a sequence having at least 97% or 98% sequence identity to SEQ ID NO: 1.
41. The nanoparticle of embodiment 39 or 40, wherein the mF8 sgRNA targets the wild-type FVIII gene sequence.
42. The nanoparticle of any of embodiments 39-41, wherein the NSGHA sgRNA includes the sequence as set forth in SEQ ID NO: 2 or a sequence having at least 97% or 98% sequence identity to SEQ ID NO: 1.
43. The nanoparticle of any of embodiments 38-42, wherein the gene-editing components include the sequence as set forth in SEQ ID NO: 11 or SEQ ID NO: 12 or have at least 90% sequence identity to the sequence as set forth in SEQ ID NO: 11 or SEQ ID NO: 12.
44. The nanoparticle of any of embodiments 38-43, wherein the nanoparticle preferentially delivers the gene-editing components to liver sinusoidal endothelial cells (LSEC).
45. The nanoparticle of any of embodiments 38-44, wherein the nanoparticle includes chondroitin sulfate, oleylamine, and sorbitan monooleate.
46. The nanoparticle of any of embodiments 38-43, wherein the nanoparticle is a lipid nanoparticle.
47. The nanoparticle of embodiment 46, wherein the lipid nanoparticle includes an ionic lipid, a helper lipid, cholesterol, and a PEG-lipid.
48. The nanoparticle of embodiment 47, wherein the ionic lipid includes 7C1, MC3, KC2, or cKK-E12.
49. The nanoparticle of embodiment 47 or 48, wherein the helper lipid includes dioleoylphosphatidylethanolamine (DOPE), 1, 2-Distearoyl-sn-glycero-3-phosphoethanolamine (DSPE), or 1,2-distearoyl-sn-glycero-3-phosphocholine (DSPC).
50. The nanoparticle of any of embodiments 47-49, wherein the cholesterol includes 20α-Hydroxycholestrol.

51. The nanoparticle of any of embodiments 47-50, wherein the PEG-lipid includes DMG-PEG2000 (1,2-Dimyristoyl-rac-glycero-3-methoxypolyethylene glycol-2000).
52. The nanoparticle of any of embodiments 38-51, wherein the lipid nanoparticle is conjugated to an LSEC targeting component.
53. The nanoparticle embodiment 52, wherein the LSEC targeting component includes mannose, an arginylglycylaspartic acid (RGD) peptide, and/or sorbitan monooleate.
54. The nanoparticle of embodiment 53, wherein the mannose is conjugated to the PEG-lipid.
55. The nanoparticle of embodiment 53 or 54, wherein the RGD peptide is conjugated to the helper lipid.
56. The nanoparticle of any of embodiments 46-55, wherein the lipid nanoparticle includes MC3, 20α-Hydroxycholestrol, DSPE, DMG-PEG2000, and RGD peptide.
57. The nanoparticle of any of embodiments 46-55, wherein the lipid nanoparticle includes MC3, 20α-Hydroxycholestrol, DSPE, DMG-PEG2000, and mannose.
58. The nanoparticle of any of embodiments 38-57, wherein the gene-editing components include a nuclease.
59. The nanoparticle of embodiment 58, wherein the nuclease includes Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, or Csf4.
60. The nanoparticle of embodiment 58, wherein the nuclease is Cas9.
61. The nanopparticle of any of embodiments 38-60, wherein the gene-editing components include a DNA repair template.
62. The nanopparticle of embodiment 61, wherein the DNA repair template encodes a functional Factor VIII protein and includes homology arms homologous to a region within 154,835,788 to 155,026,934 on the X chromosome.
63. A composition including the nanoparticle of any of embodiments 38-62 and a pharmaceutically acceptable carrier.

(vii) Experimental Examples. Introduction. Hemophilia A (HemA) is a bleeding disorder resulting from a functional deficiency of the X-linked factor VIII (FVIII) gene. Currently HemA patients are routinely infused with FVIII protein 3-4 times per week as a prophylactic treatment, which is costly and inconvenient. Gene therapy represents a very promising alternative method to treat HemA patients. The current disclosure describes use of biocompatible nanoparticle (NP) technology to deliver nucleic acids safely into targeted organs. In combination with CRISPR/Cas9 gene-editing techniques, in vivo gene editing to correct the mutant FVIII and regain the expression of functional FVIII is described.

Mouse Models. To establish a testing platform for in vivo gene correction of mutant FVIII, a colony of the Chen et al., 2020, Mol Ther Nucleic Acids, 20:534-544 mouse model was established. This HemA mouse model harbors a five nucleotides deletion in exon 1 of FVIII gene, resulting in a frameshift mutation that leads to a premature stop codon.

In addition, a B6; 129S-F8^{tm1Kaz/J} (MFVIII-16) HemA mouse model coloney (Bi et al., 1996, Blood, 88(9):3446-50, PMID:8896409) was generated by insertion of a neo cassette to replace intron 16, leading to disruption of FVIII expression.

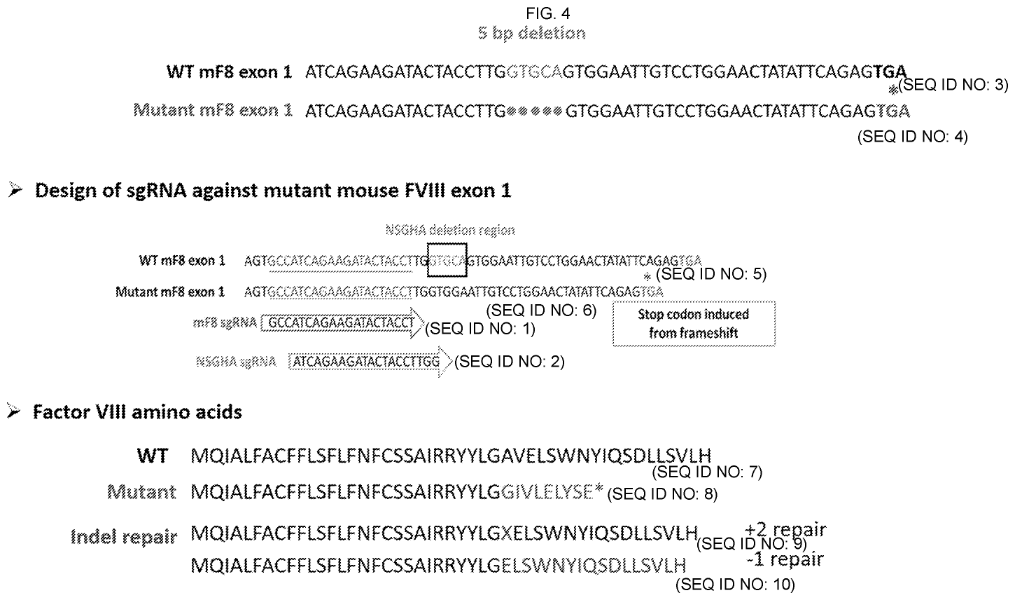

Design sgRNAs for gene editing in a specific NSG HemA mouse model. sgRNAs required for gene editing in the specific NSG HemA mouse model (with five nucleotides deletion in exon 1 of FVIII gene, leading to a premature stop codon in the NSG background) were designed and tested (FIG. 4). The sgRNA is designed to accurately target desired genome sequence and prevent any off-target editing that can result in unpredictable adverse events. Two different sgRNAs were designed; one can edit wild-type and mutant FVIII exon 1 sequence (mF8sgRNA), and the other one can only edit the mutant FVIII exon 1 sequence (NSGHAsgRNA), respectively according to CRISPR-Cas9 guide RNA design checker on the INTGRATED DNA TECHNOLOGIES (IDT) website (https://www.idtdna.com) (FIG. 4).

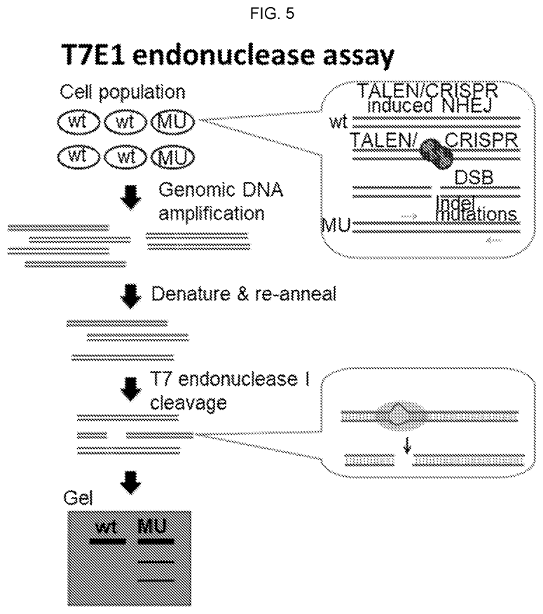

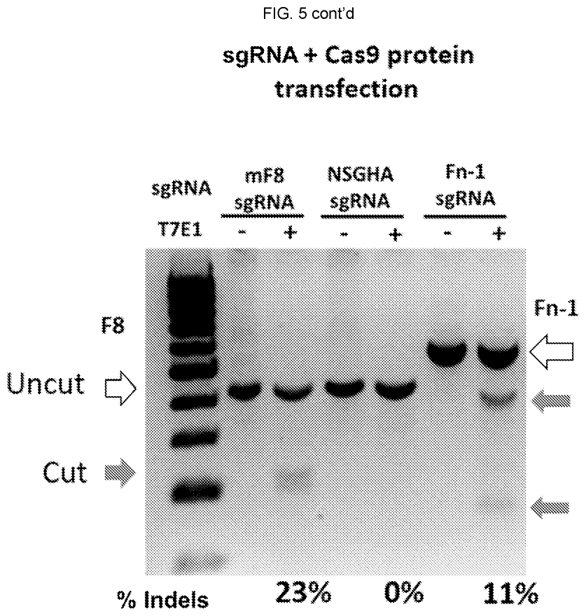

Preliminary testing of gene editing in cell culture and NSG HemA mice. The efficiency of gene editing was estimated in vitro by T7E1 assay using mouse embryonic fibroblast NIH3T3 cells. The guide RNA, mF8sgRNA was selected to target the wild-type FVIII gene in these cells. NSGHAsgRNA that only targets mutant exon 1 sequence in NSG HemA mice and sgRNA that can target mouse fibronectin exon 1 were served as controls. The Cas9/sgRNA RNP (ribonucleoprotein) complexes were prepared by premixing of Cas9 protein and sgRNA at 1/1 ratio (mol/mol). The Cas9/sgRNA RNPs were delivered to NIH3T3 cells using Lipofectamine CRISPRMAX transfection reagent. Three days after transfection, genomic DNA was extracted and the region containing targeting sequence was amplified by PCR. After denaturing PCR products by heating, the PCR products were re-annealed by subsequent slow cooling. If the DSBs are repaired by NHEJ pathway, indel mutation will result in formation of the heteroduplex structure by wild type-mutant strands pairing in targeting site. This structure will be recognized by T7 endonuclease 1 (T7E1) and cleaved. DNA gel electrophoresis can display the patterns of uncut and cut DNA bands. The frequency of indel mutation can be evaluated by the ratio between uncut and cut DNA bands. The results of T7E1 assay demonstrated that mF8sgRNA can induce indel mutation in wild type mouse FVIII exon 1 in 23% of NIH3T3 cells. Moreover, the sgRNA NSGHA showed that no indel mutation was induced indicating its specificity in mutant mouse FVIII exon 1 sequence (FIG. 5).

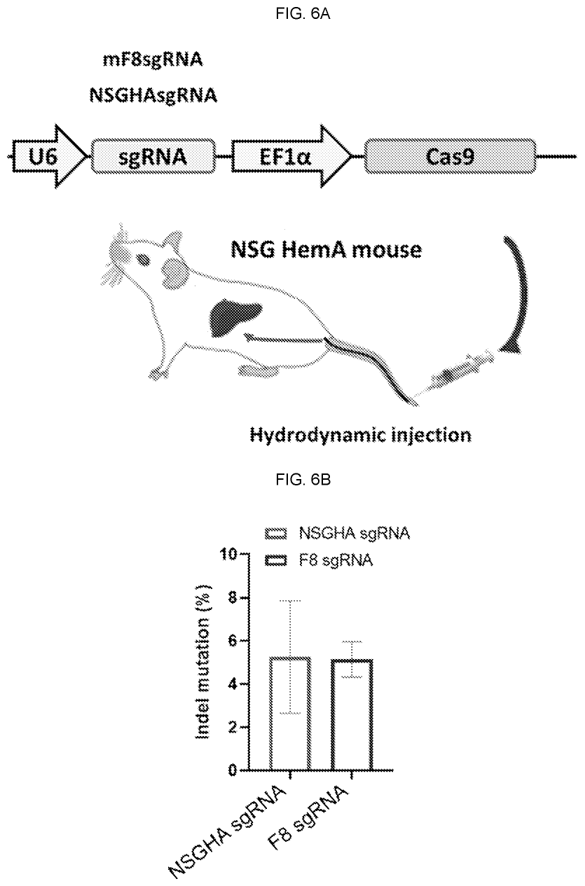

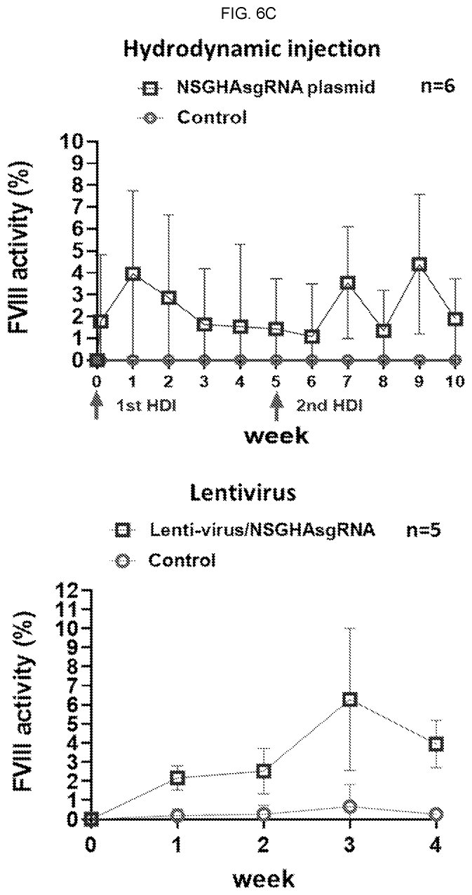

In vivo sgRNA targeting efficacy was next examined. Cas9 expression plasmids that co-expressed the specific sgRNA (NSGHAsgRNA) that targets mutant exon 1 sequence in NSG HemA mice were constructed (see FIG. 6A). The Cas9/sgRNA expression plasmids were delivered via hydrodynamic injection to the liver of NSG HemA mice. Hydrodynamic injection via the mouse tail vein can efficiently deliver pDNA into the liver and produce high level transgene expression. (Miao CH (2005) A novel gene expression system: non-viral gene transfer for hemophilia as model systems. Adv Genet, 54:143-77. doi: 10.1016/S0065-2660(05)54007-0, PMID: 16096011). The majority of the gene expression occurs in hepatocytes, however transgene expression was also observed in LSECs and Kupffer cells. The on-target indel rates were also analyzed after hydrodynamic injection of Cas9 expression plasmid that co-expressed NSGHAsgRNA and mF8sgRNA in NSG HemA mice, respectively. Similar efficiency of indel mutation was observed between these two groups of mice (FIG. 6B). The FVIII activities in treated mouse plasma were evaluated over time by activated partial thromboplastin time (aPTT). Significant elevation of FVIII activity over the course of 28 days was observed in treated mice compared to control NSG HemA mice (FIG. 6C). These significant results indicate the validity of gene editing using CRISPR/Cas9 expression and specific sgRNA to correct the mutation of FVIII gene in the specific NSG HemA mouse model.

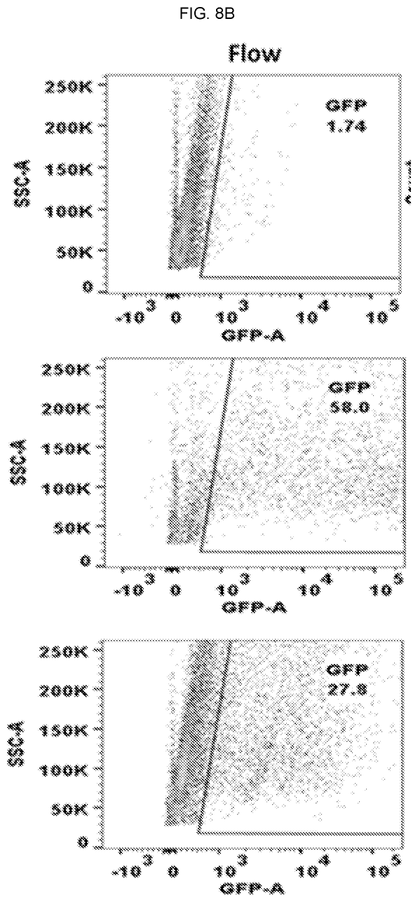

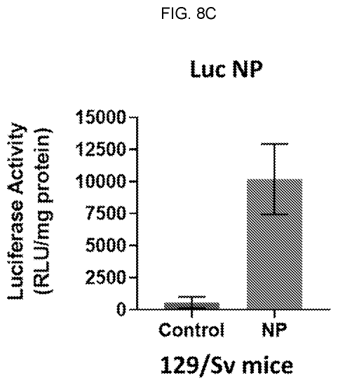

Chs-SP Nanoparticles. Since FVIII protein is mainly and naturally made in liver sinusoidal endothelial cells (LSEC), NPs were synthesized that selectively target these LSEC. DNA encapsulated NPs were synthesized using nanoprecipitation by combination of an organic phase containing chondroitin sulfate (ChS) and sorbitan ester and an aqueous phase containing p2X-GFP plasmid DNA to create ChS NPs (FIG. 7A). DNA encapsulation efficiency was examined by DNA electrophoresis (FIG. 7B). By titrating different DNA:NPs ratios, results showed that NPs carry plasmid DNA efficiently at saturating concentrations. Next, HUVEC cells were used as an endothelial cell model to evaluate the transfection efficiency of DNA encapsulated ChS-SP NPs. The transfected cells were analyzed by flow cytometry after transfection using ChS-SP NPs carrying p2X-GFP plasmid. GFP expression was detected in 26% of the cells, indicating ChS-SP NPs/DNA successfully transfect HUVEC cells (see FIGS. 8A-8C).

The data described show high transfection efficiency of DNA encapsulated NPs in HUVEC cells. Furthermore, in vivo gene editing using CRISPR/Cas9 technology to correct the mutated FVIII gene and regain the expression of functional FVIII protein in NSG HA mice was shown. NPs that carry Cas9/sgRNA plasmid can be used to correct the mutant FVIII gene in NSG HA mice.

Synthesis of LNPs to deliver mRNA into endothelial cells. LNPs typically consist of an ionic lipid, a helper lipid, cholesterol and a PEG-modified lipid (PEGylated lipid or PEG-lipid). The ionic lipids containing cationic charges can promote interaction with negatively-charged mRNA for encapsulation. Additionally, they also facilitate interaction with anionic plasma membrane of targeting cells for endocytosis and enable mRNA escape from endosome.

More particularly, compared to earlier versions of lipid polyplexes, lipid nanoparticles (LNPs) have been demonstrated to have superior stability, structural plasticity and enhanced gene delivery capabilities. A typical LNP formulation is composed of pH-responsive lipids or cationic lipids bearing tertiary or quaternary amines to encapsulate the polyanionic mRNA. Helper lipids are usually neutral lipids like dioleoyl phosphatidyl ethanolamine (DOPE) or 1,2-distearoyl-sn-glycero-3-phosphocholine (DSPC), and/or sterol lipids such as cholesterol to stabilize the lipid bilayer of the LNP and to enhance mRNA delivery efficiency. Polyethylene glycol (PEG)-lipid is used to improve the colloidal stability in a biological environment such as in blood circulation by reducing the specific absorption of plasma proteins and forming a hydration layer over the nanoparticles. The morphology of lipid nanoparticles is not like a traditional liposome, characterized by a lipid bilayer surrounding an aqueous core. LNPs possess an electron-dense core where the cationic/ionizable lipids are organized into inverted micelles around the encapsulated mRNA molecules.

Most LNPs developed for delivery of nucleic acids to the liver predominantly delivers to hepatocytes. In a previous study (Chen et al., 2020, Mol Ther Nucleic Acids, 20:534-544. doi: 10.1016/j.omtn.2020.03.015, PMC7178004, PMID:32330871), luciferase (Luc) mRNA was successfully delivered to obtain high expression of luciferase in mice. Afterwards, different tissues/organs were harvested from the treated mice and imaged for luciferase signal. Luciferase expression was mainly detected in the liver and weak or no expression was observed in spleen and other organs, suggesting LNPs delivered mRNA to the liver predominantly. Furthermore, immune-fluorescent staining of luciferase protein was carried out in liver of treated mice using endothelial markers and the nuclear markers. Strong luciferase signal (red) was detected homogeneously in hepatocytes, whereas no luciferase signal (red) overlaid with endothelial marker vWF signal (green). The results clearly demonstrated that these Luc LNPs can deliver mRNA efficiently into the liver, however predominantly targeted hepatocytes rather than endothelial cells. However, LSECs are the primary site of native FVIII synthesis. Thus, it is optimal to deliver gene editing tools to LSECs to achieve effective correction of FVIII gene.

(6Z,9Z,28Z,31Z)-heptatriacont-6,9,28,31-tetraene-19-yl 4-(dimethylamino) butanoate (DLin-MC3-DMA, or abbreviated to MC3) was originally reported in 2010. (Jayaraman et al., 2012, Angew Chem Int Ed Engl, 51(34):8529-33. doi: 10.1002/anie.201203263, PMC3470698, PMID:22782619). MC3-based LNP can safely deliver mRNA or siRNA in both mice and humans and is one of the gold standard cationic lipids for clinical liver targets. Moreover, MC3-based LNPs have been taken up not only by hepatocyte but also Kupffer cells and LSECs in liver. (Sago et al., 2019, Cellular and Molecular Bioengineering, 12(5):389-397. doi:10.1007/s 12195-019-00573-4). In addition, several potential ionic lipid-based LNPs are reported to potentially target LSECs.

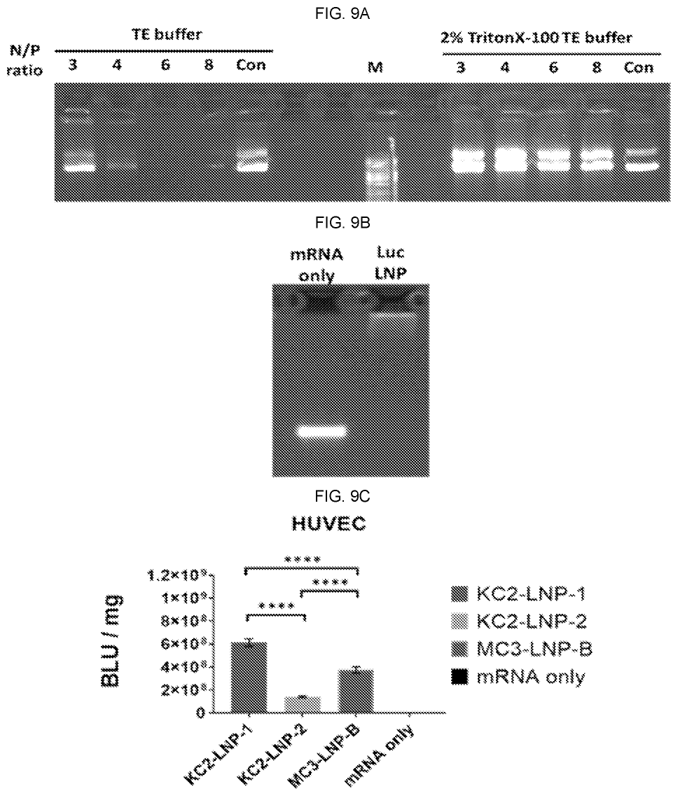

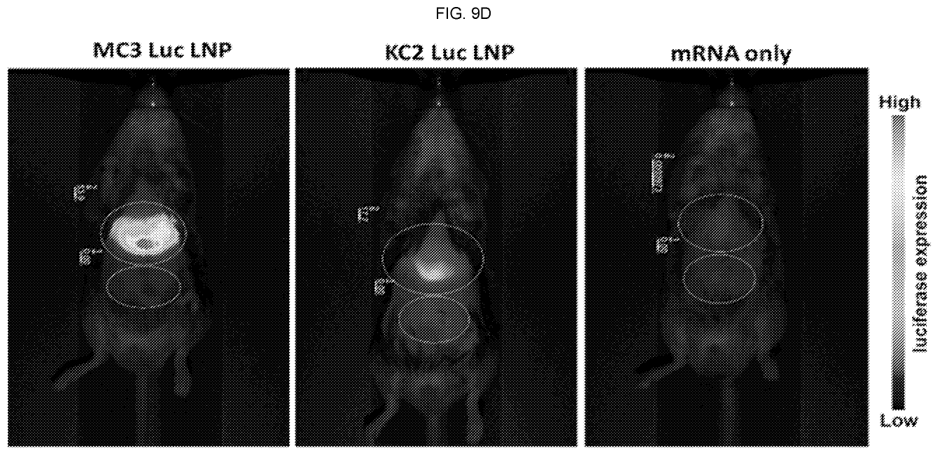

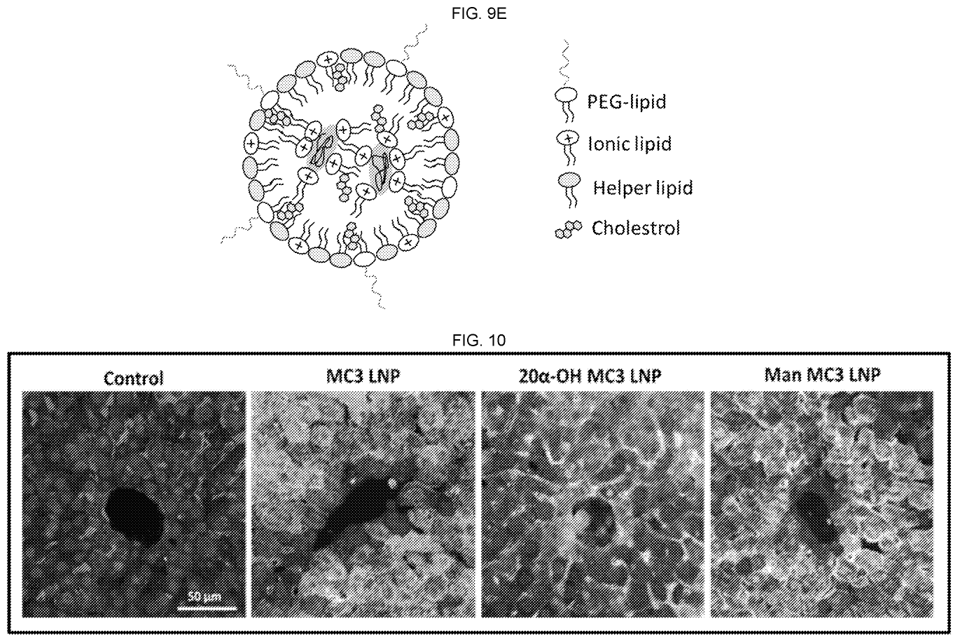

After screening of DLinDMA-based lipids, DLin-KC2-DMA (KC2) was demonstrated to have the best performance of in vivo activity in liver in mice and non-human primates. Since MC3- and KC2-based LNPs were shown to deliver mRNA not only to hepatocytes, but also endothelial cells, Luc mRNA was first encapsulated into MC3-based LNPs. Briefly, ionic lipid MC3 was mixed with the helper lipid 1,2-dioleoyl-sn-glycero-3-phosphoethanolamine (DOPE), cholesterol (Chol) and 1,2-dimyristoyl-rac-glycero-3-methoxypolyethylene glycol-2000 (DMG-PEG2000) in ethanol (MC3/DOPE/Chol/DMG-PEG2000=50/10/39/1, mole ratio). The lipid components were formulated with nucleic acid at a volume ratio of 1:3 and assembled by NanoAssemblr system (Precision NanoSystems) to synthesize Luc mRNA encapsulated LNPs (MC3-Luc LNP-B). After replacement of ethanol with PBS and concentrating mRNA using Amicon Ultra filter units, the efficiency of nucleic acid encapsulating into LNPs was identified by gel electrophoresis. Nitrogen and phosphate ratio (N/P) between ionic lipid and pDNA was titrated to improve the encapsulation efficiency. Higher N/P (6 and 8) resulted in good encapsulation efficiency without leaky pDNA when resuspended in TE buffer (FIG. 9A). The optimized N/P ratio was used to synthesize Luc mRNA LNP. FIG. 9B showed that no free mRNA was present, indicating high encapsulation efficiency of mRNA in LNPs.

To investigate the delivery efficiency of LNPs, the transfection efficiency of Luc LNPs in a primary endothelial cell line-Human Umbilical Vein Endothelial Cells (HUVEC) was tested. The result suggested that HUVECs can take up MC3-Luc LNP-B and express luciferase protein (FIG. 9C). Different ionic lipid KC2-based LNPs were also tested using similar helper lipids and molar ratios (KC2/DOPE/Chol/DMG-PEG2000 =50/10/39/1). KC2-LNP-1 encapsulated Luc mRNA in 50 mM sodium citrate buffer (pH3.0), whereas KC2-LNP-2 encapsulated Luc mRNA in 25mM NaOAc buffer (pH4.0). All three LNPs were able to deliver Luc mRNA into HUVEC (FIG. 9C), whereas no luciferase expression was detected in control cells with mRNA only.

In a test of these new LNPs for in vivo gene delivery, two groups of mice were intravenously injected with two different formulations of LNPs (MC3-LNP-B and KC2-LNP-1) encapsulated with luciferase mRNA, respectively (0.3 mg/kg). The control group was injected with mRNA only. The luciferase expression was evaluated using Pearl in vivo image system (IVIS) at 4 fours after LNP treatment. Mice injected with Luc LNPs showed significant bioluminescence signals compared with mRNA only treated mice (FIG. 9D). These results demonstrate that both MC3- and KC-based LNPs are capable of delivering mRNA in vivo and targeting LSECs efficiently.

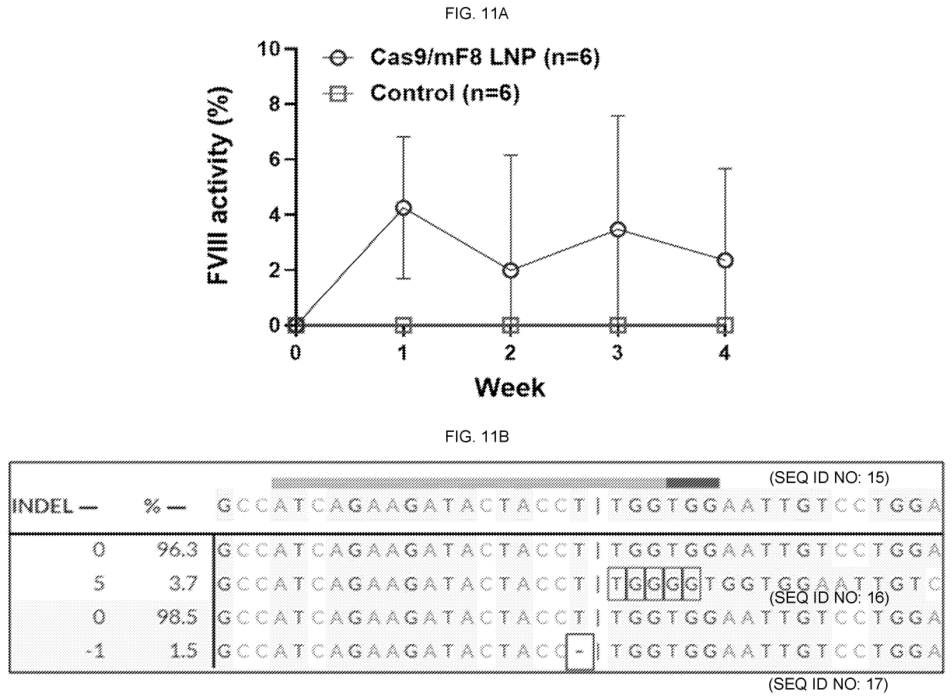

Delivery of mRNA into the mouse liver using different formulation of MC3-based LNPs. To enhance LSEC targeting, in vivo studies using different formulations of MC3-based LNPs were performed. Mice were intravenously injected with three formulations of GFP mRNA LNPs including basic MC3-based LNP formulation (MC3 LNP), 20α-hydroxycholesterol MC3 LNP (20α-OH MC3 LNP) and mannose conjugated MC3 LNP (Man MC3 LNP), respectively (0.6-0.88 mg/kg). The GFP expression was investigated using immunostaining at 6 fours after LNP treatment. Mice injected with GFP MC3 LNPs showed GFP expression predominantly in hepatocytes with minor expression in LSECs. Interestingly, mice treated with 20α-OH MC3 LNP or Man MC3 LNP showed much more distribution of GFP signal in LSECs compared to the basic MC3 formulation (FIG. 10), indicating enhancement of LSEC-targeted delivery by incorporating modified lipid components to MC3 LNPs.

Cas9 mRNA/mF8 sgRNA LNP can induce indel mutation in NSG HemA mice in vivo. The in vivo gene targeting efficacy of LNPs carrying Cas9 mRNA and sgRNA (Cas9/sgRNA LNP) was examined. Both Cas9 mRNA and mF8sgRNA were encapsulated into MC3 LNP simultaneously and intravenously injected into NSG HemA mice. FVIII activities were evaluated over time by aPTT. 2.4±0.9% (the highest level up to 10.4%) FVIII activity was observed after Cas9/mF8sgRNA LNP treatment compared to untreated control NSG HemA mice (FIG. 11A). Liver genomic DNA isolated from treated mice was subjected to sequencing analysis. In one mouse, deletion of one base pair and insertion of five base pairs in the target region were found, all of which led to in-frame correction of the FVIII coding sequence (FIG. 11B). Various insertion/deletion patterns in the target site with or without correcting the frame shift were also found in other treated mice. These exciting results demonstrate the use of FVIII gene repair using Cas9/sgRNA LNPs.

(viii) Closing Paragraphs. The nucleic acid and amino acid sequences provided herein are shown using letter abbreviations for nucleotide bases and amino acid residues, as defined in 37 C.F.R. § 1.822 and set forth in the tables in WIPO Standard ST.25 (1998), Appendix 2, Tables 1 and 3. Only one strand of each nucleic acid sequence is shown, but the complementary strand is understood as included in embodiments where it would be appropriate.

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (Ile), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, Ile, Phe, Met, and Trp; Group 11 (aliphatic): Gly, Ala, Val, Leu, and Ile; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like. As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y.. Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridize under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5XDenhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50 ° C. with 1×SSPE, 0.1% SDS. In addition, to achieve even lower stringency, washes performed following stringent hybridization can be done at higher salt concentrations (e.g., 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

“Specifically binds” refers to an association of a binding domain to its cognate binding molecule with an affinity or Ka (i.e., an equilibrium association constant of a particular binding interaction with units of 1/M) equal to or greater than 10⁵ M⁻¹, while not significantly associating with any other molecules or components in a relevant environment sample. “Specifically binds” is also referred to as “binds” herein. Binding domains may be classified as “high affinity” or “low affinity”. In particular embodiments, “high affinity” binding domains refer to those binding domains with a Ka of at least 107 M-1, at least 108 M-1, at least 109 M-1, at least 1010 M-1, at least 1011 M-1, at least 1012 M-1, or at least 1013 M-1. In particular embodiments, “low affinity” binding domains refer to those binding domains with a Ka of up to 107 M-1, up to 106 M-1, up to 105 M-1. Alternatively, affinity may be defined as an equilibrium dissociation constant (Kd) of a particular binding interaction with units of M (e.g., 10-5 M to 10-13 M). In certain embodiments, a binding domain may have “enhanced affinity,” which refers to a selected or engineered binding domains with stronger binding to a cognate binding molecule than a wild type (or parent) binding domain. For example, enhanced affinity may be due to a Ka (equilibrium association constant) for the cognate binding molecule that is higher than the reference binding domain or due to a Kd (dissociation constant) for the cognate binding molecule that is less than that of the reference binding domain, or due to an off-rate (Koff) for the cognate binding molecule that is less than that of the reference binding domain. A variety of assays are known for detecting binding domains that specifically bind a particular cognate binding molecule as well as determining binding affinities, such as Western blot, ELISA, and BIACORE® analysis (see also, e.g., Scatchard, et al., 1949, Ann. N.Y. Acad. Sci. 51:660; and U.S. Pat. Nos. 5,283,173, 5,468,614, or the equivalent).

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the ability to obtain a claimed effect according to a relevant experimental method described in the current disclosure.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A method of treating hemophilia A in a subject in need thereof, the method comprising administering a therapeutically effective amount of an LSEC-targeted nanoparticle to the subject, wherein the LSEC-targeted nanoparticle is associated with (i) SEQ ID NO: 1 and (ii) a nuclease and/or a nucleotide sequence encoding the nuclease
and wherein the nanoparticle comprises:
an ionic lipid comprising MC3 or KC2;
a helper lipid comprising dioleoylphosphatidylethanolamine (DOPE), 1,2-Distearoyl-sn-glycero-3-phosphoethanolamine (DSPE), or 1,2-distearoyl-sn-glycero-3-phosphocholine (DSPC);
cholesterol;
a polyethylene glycol (PEG)-lipid; and
an LSEC targeting ligand comprising mannose and/or arginylglycylaspartic acid (RGD) peptide, wherein the ionic lipid, helper lipid, cholesterol, and PEG-lipid are present within the nanoparticle at a ratio of about 50/10/39/1.

2. A method of preferentially genetically-modifying the Factor VIII gene in liver sinusoidal endothelial cells (LSEC) in a subject, the method comprising
administering a therapeutically effective amount of an LSEC-targeted nanoparticle to the subject, wherein the LSEC-targeted nanoparticle is associated with (i) a nucleotide sequence having the sequence as set forth in SEQ ID NO: 1 or having at least 97% sequence identity to the sequence as set forth in SEQ ID NO: 1 and (ii) a nuclease and/or a nucleotide sequence encoding the nuclease and wherein the nanoparticle comprises:
an ionic lipid, a helper lipid, cholesterol, a polyethylene glycol (PEG)-lipid, and an LSEC targeting ligand comprising mannose and/or arginylglycylaspartic acid (RGD) peptide or wherein the nanoparticle comprises chondroitin sulfate, oleylamine, and sorbitan monooleate.

3. A method of claim 2, wherein the ionic lipid, helper lipid, cholesterol, and PEG-lipid are present within the nanoparticle at a ratio of about 50/10/39/1.

4. The method of claim 2, wherein the ionic lipid comprises MC3, KC2, 7C1, or cKK-E12.

5. The method of claim 2, wherein the cholesterol comprises 20α-Hydroxycholestrol.

6. The method of claim 2, wherein the PEG-lipid comprises 1,2-Dimyristoyl-rac-glycero-3-methoxypolyethylene glycol-2000 (DMG-PEG2000).

7. The method of claim 2, wherein the nanoparticle is associated with a nucleic acid sequence as set forth in SEQ ID NO: 11 or SEQ ID NO: 12 or a sequence having at least 90% sequence identity to the sequence as set forth in SEQ ID NO: 11 or SEQ ID NO: 12.

8. The method of claim 2, wherein the nanoparticle is associated with a nucleic acid sequence as set forth in SEQ ID NO: 11 or SEQ ID NO: 12 or a sequence having at least 95% sequence identity to the sequence as set forth in SEQ ID NO: 11 or SEQ ID NO: 12.

9. The method of claim 2, wherein the subject has been diagnosed with hemophilia A.

10. The method of claim 2, wherein the therapeutically effective amount results in expression of functional Factor VIII within the subject.

11. The method of claim 10, wherein the expression of the functional Factor VIII ameliorates a symptom of hemophilia A in the subject.

12. The method of claim 2, wherein the subject is murine or human.

13. The method of claim 2, wherein the nuclease is selected from the group consisting of Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, or Csf4.

14. The method of claim 2, wherein the nuclease is Cas9.

15. The method of claim 2, wherein the nucleotide sequence encoding the nuclease is in the form of plasmid DNA.

16. The method of claim 2, wherein the LSEC-targeted lipid nanoparticle comprises mannose conjugated to the PEG-lipid.

17. The method of claim 2, wherein the LSEC-targeted lipid nanoparticle comprises the RGD peptide conjugated to the helper lipid.

18. The method of claim 2, wherein the lipid nanoparticle comprises MC3, 20α-Hydroxycholestrol, DSPE, DMG-PEG2000, and RGD peptide.

19. The method of claim 2, wherein the lipid nanoparticle comprises MC3, 20α-Hydroxycholestrol, DSPE, DMG-PEG2000, and mannose.

20. The method of claim 2, wherein the lipid nanoparticle comprises KC2, 20α-Hydroxycholestrol, DSPE, DMG-PEG2000, and RGD peptide.

21. The method of claim 2, wherein the lipid nanoparticle comprises KC2, 20α-Hydroxycholestrol, DSPE, DMG-PEG2000, and mannose.

22. The method of claim 2, further comprising delivering a DNA repair template.

23. The method of claim 22, wherein the DNA repair template encodes a functional Factor VIII protein.

24. The method of claim 23, wherein the functional Factor VIII protein has the sequence as set forth in SEQ ID NO: 13 or SEQ ID NO: 14.

25. The method of claim 23, wherein the functional Factor VIII protein has at least 95% sequence identity to the sequence as set forth in SEQ ID NO: 13 or SEQ ID NO: 14.

26. The method of claim 22, wherein the DNA repair template is encoded by a viral vector.

27. The method of claim 26, wherein the viral vector is an adeno associated viral vector.

28. The method of claim 2, wherein the administering is through intravenous, intradermal, intraarterial, intranodal, intravesicular, intrathecal, intraperitoneal, intraparenteral, intranasal, intralesional, intramuscular, inhaled, or subcutaneous administration.

29. The method of claim 2, wherein the administering is through intravenous administration.

30. The method of claim 2, wherein the administering comprises administering multiple doses of the therapeutically effective amount.

31. A nanoparticle associated with (i) a nucleic acid having the sequence as set forth in SEQ ID NO: 1 or SEQ ID NO: 2 or having a sequence with at least 97% sequence identity to the sequence as set forth in SEQ ID NO: 1 or SEQ ID NO: 2, and (ii) a liver sinusoidal endothelial cells (LSEC) targeting agent.

32. The nanoparticle of claim 31, associated with a nucleic acid having the sequence as set forth in SEQ ID NO: 1.

33. The nanoparticle of claim 31, associated with a nucleic acid having the sequence as set forth in SEQ ID NO: 2.

34. The nanoparticle of claim 31, associated with a nucleic acid having the sequence as set forth in SEQ ID NO: 11 or SEQ ID NO: 12 or a sequence with at least 90% sequence identity to the sequence as set forth in SEQ ID NO: 11 or SEQ ID NO: 12.

35. The nanoparticle of claim 31, wherein the nanoparticle is a lipid nanoparticle.

36. The nanoparticle of claim 35, wherein the lipid nanoparticle comprises an ionic lipid, a helper lipid, cholesterol, and a PEG-lipid.

37. The nanoparticle of claim 35, wherein the ionic lipid comprises MC3, KC2, 7C1, or cKK-E12.

38. The nanoparticle of claim 36, wherein the helper lipid comprises dioleoylphosphatidylethanolamine (DOPE), 1,2-Distearoyl-sn-glycero-3-phosphoethanolamine (DSPE), or 1,2-distearoyl-sn-glycero-3-phosphocholine (DSPC).

39. The nanoparticle of claim 36, wherein the cholesterol comprises 20α-Hydroxycholestrol.

40. The nanoparticle of claim 36, wherein the PEG-lipid comprises 1,2-Dimyristoyl-rac-glycero-3-methoxypolyethylene glycol-2000 (DMG-PEG2000).

41. The nanoparticle of claim 31, wherein the nanoparticle comprises chondroitin sulfate, oleylamine, and sorbitan monooleate.

42. The nanoparticle of claim 31, wherein the LSEC targeting agent comprises mannose and/or arginylglycylaspartic acid (RGD) peptide.

43. The nanoparticle of claim 42, wherein the mannose is conjugated to the PEG-lipid.

44. The nanoparticle of claim 42, wherein the RGD peptide is conjugated to the helper lipid.

45. The nanoparticle of claim 42, wherein the lipid nanoparticle comprises MC3, 20α-Hydroxycholestrol, DSPE, DMG-PEG2000.

46. The nanoparticle of claim 45, wherein the MC3, 20α-Hydroxycholestrol, DSPE, DMG-PEG2000 are present within the nanoparticle at a ratio of about 50/10/39/1.

47. The nanoparticle of claim 46, further comprising an RGD peptide.

48. The nanoparticle of claim 46, further comprising mannose.

49. The nanoparticle of claim 35, wherein the lipid nanoparticle comprises KC2, 20α-Hydroxycholestrol, DSPE, DMG-PEG2000.

50. The nanoparticle of claim 49, wherein the KC2, 20α-Hydroxycholestrol, DSPE, DMG-PEG2000 are present within the nanoparticle at a ratio of about 50/10/39/1.

51. The nanoparticle of claim 50, further comprising an RGD peptide.

52. The nanoparticle of claim 50, further comprising mannose.

53. The nanoparticle of claim 31, wherein the nanoparticle is further associated with a nuclease or a nucleic acid encoding a nuclease.

54. The nanoparticle of claim 53, wherein the nuclease comprises Cas1, Cas1B, Cas2, Cas3, Cas4, Cas5, Cas6, Cas7, Cas8, Cas9, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, or Csf4.

55. The nanoparticle of claim 53, wherein the nuclease comprises Cas9.

56. A composition comprising the nanoparticle of claim 31 and a pharmaceutically acceptable carrier.
